(19) United States
(12) Patent Application Publication
(10) Pub. No.: US 2012/0041069 A1
(43) Pub. Date: Feb. 16, 2012

(54) SGLT2 INHIBITOR DOSAGE FORMS

(75) Inventor: Ramesh SESHA, West Windsor, NJ (US)

(73) Assignee: Nectid Inc.

(21) Appl. No.: 13/089,068

(22) Filed: Apr. 18, 2011

Related U.S. Application Data
(63) Continuation of application No. PCT/US2009/061201, filed on Nov. 19, 2009.

(51) Int. Cl. A61K 31/155; A61P 3/10

(52) U.S. Cl. 514/635

(57) ABSTRACT

A pharmaceutical composition comprising a sodium-dependent glucose transporter (SGLT2) inhibitor and a biguanide, wherein at least one of the active agents is in slow release form, is provided. A method for treating diabetes in a patient in need thereof including administering an anti-diabetic combination comprising a sodium-dependent glucose transporter (SGLT2) inhibitor and a biguanide, wherein at least one of the active agents is in slow release form, is also provided.

### RELATED APPLICATION

This application is a continuation of international patent application PCT/US2009/061201, filed Oct. 19, 2009, and published as WO 2010/045656 A2; which claims priority from U.S. provisional patent application Ser. No. 61/196,369, filed on Oct. 17, 2008, which are incorporated herein by reference.

Diabetes mellitus is a progressive metabolic disorder with diverse pathologic manifestations and is often associated with lipid metabolism and glycometabolic disorders. The long-term effects of diabetes result from its vascular complications, e.g., the microvascular complications of retinopathy, neuropathy and nephropathy, and the macrovascular complications of cardiovascular, cerebrovascular and peripheral vascular diseases. Initially, diet and exercise is the mainstay of treatment of type II diabetes. However, this is often followed by administration of oral hypoglycemic agents.

Exemplary drugs useful for managing type II diabetes and its precursor syndromes such as insulin resistance include different classes of compounds, biguanides such as metformin, phenformin, buformin, sulfonylureas such as glipizide, glimiperide, glyburide, glibornuride, glisoxepide, gliclazide acetohexamide, chlorpropamide, tolazamide, and tolbutamide PPAR agents such as troglitazone, pioglitazone, rosiglitazone, ciglitazone, isaglitazone, darglitazone, zorglitazone, englitazone, balaglitazone etc, α-glycosidase inhibitors such as acarbose and miglitol, meglitinides such as repaglinide, nateglinide, Dual PPAR agonists such as aleglitazar, muraglitazar, tesaglitazar etc, Dipeptidyl Peptidase IV inhibitors (DPP IV inhibitors) such as sitagliptin, vildagliptin, alogliptin, saxagliptin, dutogliptin, linagliptin, melogliptin etc, Glucagon-like peptide-1 analogs such as exenatide, liraglutide, albiglutide, taspoglutide etc. Exemplary structures of each of these classes of anti-diabetic drugs are listed below. At least one drug in each class of agents has been approved while a large number of others are in the pipeline.

Sodium-dependent glucose transporter (SGLT2) inhibitor such as Dapagliflozin (IUPAC name: 2S, 3R, 4R, 5S, 6R)-2-[4-chloro-3-(4-ethoxybenzyl)phenyl]-6-(hydroxymethyl) tetrahydro-2H-pyran-3,4,5-triol, Molecular Weight; 503), Remogliflozin (β-D-Glucopyranoside, 5-methyl-4[[4-(1-methylethoxy)phenyl]methyl]-1-(1-methylethyl)-1H-pyrazol-3-yl, 6-(ethyl carbonate, Molecular Weight: 522), Sergliflozin (IUPAC name; 2-[(4-methoxyphenyl) methyl]phenyl 6-O-(ethoxycarbonyl)-β-D-glucopyranoside, Molecular weight: 448), JNJ 28431754/TA-7284 (Canagliflozin), ISIS 388626, BI 10773, BI 44847, and AVE 2268 etc, are another novel class of anti-diabetic agents that are under clinical trials for the treatment of diabetes. Sodium-dependent glucose co-transporters are a family of glucose transporter found in the intestinal mucosa of the small intestine (SGLT1) and the proximal tubule of the nephron (SGLT2 and SGLT1). They contribute to renal glucose re-absorption. Dapagliflozin or its pharmaceutically acceptable salts or solvates thereof (hereinafter dapagliflozin), an orally active sodium-dependent glucose transporter (SGLT2) inhibitor is disclosed in U.S. Pat. No. 6,515,117. The molecular structures of representative examples of sodium-dependent glucose transporter (SGLT2) inhibitors are below.

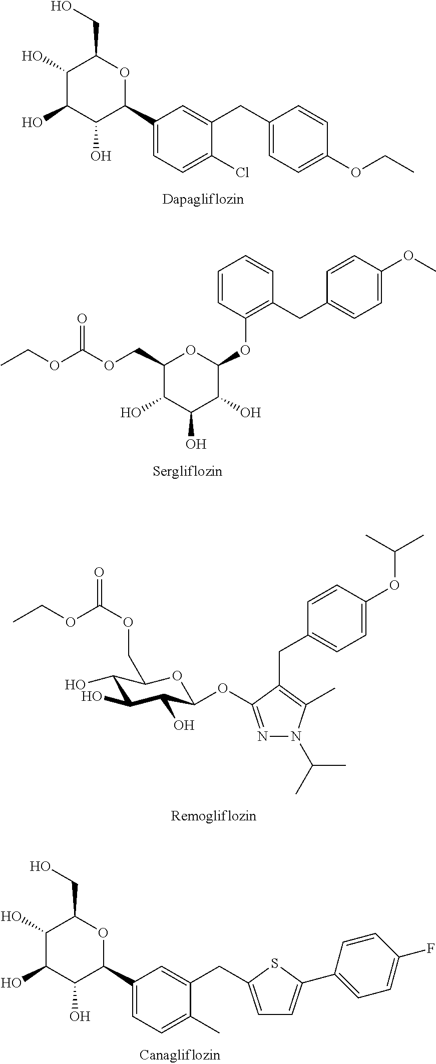

Presently, DPP IV inhibitors, biguanides, glitazones and sulfonylureas are commercially available in the form of tablets of the individual drugs, in immediate release (IR) formulations or in controlled release (CR) formulations. These are usually administered orally to patients in need thereof, using protocols calling for the administration of the individual ingredient.

In type 2 diabetic patients failure of monotherapy manifests itself in the form of Insulin resistance and reduced insulin secretion. Therefore, treatment approaches include reducing insulin resistance or increasing insulin sensitivity and augmenting insulin secretion from the pancreatic beta cells. The tissues most commonly resistant to the actions of insulin are liver, skeletal muscles, and adipose tissues. Therefore, combination treatment strategies directed towards improving the insulin sensitivity of these major tissues can help the patients.

Typically, metformin monotherapy has been used as the initial treatment in diabetic patients. If monotherapy fails it may be supplemented with other drugs. One solution for treating T2DM uses at least two drugs to obviate the mono-therapy difficulties that can accompany prolonged use of metformin. The addition of a second drug, e.g., DPP IV inhibitors, glitazones or sulfonylureas to the concurrent treatment can provide a balance of stimulated release of insulin while ameliorating insulin resistance. This can provide an optimal level of glycemic control that is unattainable using monotherapy. However, requiring a patient to take multiple medications for the prophylaxis or treatment of diseases can result in patient inconvenience and lead to non-compliance of the prescribed dosage regimen. The ease of using single composition for multiple medications as opposed to separate administration of the individual medications has long been recognized in the practice of medicine. Such a composition can provide a therapeutic advantage for the benefit of the patient and the clinician. Further, such a composition can provide both increased convenience and improved patient compliance resulting from the avoidance of missed doses through patient forgetfulness.

Pharmaceutical dosage forms containing combinations of anti-diabetic drugs are known from for example, EPO 0 749 751 discloses pioglitazone as an insulin sensitivity enhancer, combined with other anti-diabetics such as metformin, phenformin or buformin. The '751 application also discloses that these drugs can be associated (mixed or coated) with conventional excipients to provide taste masking or provide a sustained or slow release. U.S. Pat. No. 6,011,049 discloses a pharmaceutical composition having pioglitazone or trolitazone and metformin in slow release forms such as osmotic pumps or skin patches. Other combinations of antihyperglycemic drugs and thiazolidinedione derivatives can be found, e.g., in U.S. Pat. Nos. 6,524,621; 6,475,521; 6,451,342 and 6,153,632 and PCT patent applications WO 01/3594 and WO 01/3594. U.S. Pat. No. 7,125,873 discloses a pharmaceutical composition of a DPP IV inhibitor, e.g., Sitagliptin with other anti-diabetic drugs such as biguanide and PPAR agonists. U.S. Patent Application No. 20090105265, discloses pharmaceutical compositions comprising fixed-dose combinations of a dipeptidyl peptidase-4 inhibitor and metformin, methods of preparing such pharmaceutical compositions, and methods of treating Type 2 diabetes with such pharmaceutical compositions. U.S. Patent Application No. 20080234366 discloses pharmaceutical formulations are provided which are in the form of capsules or tablets for oral use and which include a medicament such as dapagliflozin and a pharmaceutical acceptable carrier, which is designed for immediate release.

There is a need for pharmaceutical compositions comprising multiple drugs, e.g., a sodium-dependent glucose transporter (SGLT2) inhibitor and a slow release biguanide. Further, there is a need for a method for administering the combination of a slow release biguanide and a sodium-dependent glucose transporter (SGLT2) inhibitor that provides the advantages discussed above.

### DESCRIPTION

The present invention provides, in one aspect, pharmaceutical compositions comprising a sodium-dependent glucose transporter (SGLT2) inhibitor and a biguanide, wherein at least one of the active agents is in slow or controlled release form. The compositions can provide continuous and non-pulsating therapeutic levels of said biguanide to a mammal e.g., a human, in need of such treatment over about an eight-hour to about a twenty-four hour period. Non-limiting examples of sodium-dependent glucose transporter (SGLT2) inhibitors include Dapagliflozin, Sergliflozin and Remogliflozin and the like. Non-limiting examples of biguanides include metformin, phenformin, or buformin, and pharmaceutically acceptable salts thereof.

In another aspect, the invention provides pharmaceutical compositions comprising a slow release biguanide, a slow release sodium-dependent glucose transporter (SGLT2) inhibitor, and optionally at least one pharmaceutically acceptable excipient and an immediate release layer comprising a sodium-dependent glucose transporter (SGLT2) inhibitor that can provide continuous and non-pulsating therapeutic levels of a biguanide drug to an animal in need of such treatment over a twelve hour or twenty-four hour period.

In yet another aspect, the invention provides a pharmaceutical combination comprising a slow release metformin, a slow release sodium-dependent glucose transporter (SGLT2) inhibitor, and optionally at least one pharmaceutically acceptable excipient and an immediate release layer comprising a sodium-dependent glucose transporter (SGLT2) inhibitor that can provide continuous and non-pulsating therapeutic levels of metformin to an animal in need of such treatment over a twelve hour or twenty-four hour period.

In yet another aspect, the invention also provides a pharmaceutical composition comprising a slow release biguanide, e.g., metformin, a slow release sodium-dependent glucose transporter (SGLT2) inhibitor and optionally at least one pharmaceutically acceptable excipient, wherein the composition is useful for treating diabetes.

In yet another aspect, the invention also provides a pharmaceutical composition comprising a slow release sodium-dependent glucose transporter (SGLT2) inhibitor, an immediate release biguanide, e.g., metformin, and optionally at least one pharmaceutically acceptable excipient that is useful for treating diabetes.

In yet another aspect the invention provides a method for administering a pharmaceutical composition comprising a sodium-dependent glucose transporter (SGLT2) inhibitor and a biguanide, to a patient in need thereof, wherein at least one of the active agents is in slow release form. The composition can include a sodium-dependent glucose transporter (SGLT2) inhibitor, such as, for example, Dapagliflozin, Sergliflozin, Remogliflozin, ISIS 388626, JNJ 28431754/TA-7284, BI 10773, BI 44847, and AVE 2268 and the like, and biguanides such as, for example, metformin, phenformin, or buformin or pharmaceutically acceptable salts thereof.

In yet another aspect the invention provides a method for treating diabetes comprising administering, to a patient in need thereof, a composition which can include a sodium-dependent glucose transporter (SGLT2) inhibitor, such as, for example, Dapagliflozin, Sergliflozin, Remogliflozin, ISIS 388626, JNJ 28431754/TA-7284, BI 10773, BI 44847, and AVE 2268 and the like, and biguanides such as metformin, phenformin, or buformin, and pharmaceutically acceptable salts thereof.

In another aspect, the present invention provides a dosage form that can deliver a sodium-dependent glucose transporter (SGLT2) inhibitor and a biguanide wherein the peak plasma levels of the biguanide compound is approximately 8-12 hours after administration and peak plasma levels of a SGLT2 inhibitor is approximately 1-4 hours after dosing.

In another aspect of this invention provides a pharmaceutical composition comprising a slow release metformin portion and an sodium-dependent glucose transporter (SGLT2) inhibitor, wherein the said composition exhibits a dissolution profile such that after about two hours from 0 to about 25 percent of the metformin is released, after about four hours from about 10 to about 45 percent of the metformin is released, after eight hours from about 30 to about 90 percent of metformin is released. In another aspect, after about twelve hours at least about 50 percent of the metformin is released, and after about sixteen hours at least about 60 percent of the metformin is released.

In another aspect, the invention provides a combination comprising a slow release biguanide, e.g., metformin, and a slow release sodium-dependent glucose transporter (SGLT2) inhibitor, and optionally at least one pharmaceutically acceptable excipient and an immediate release layer comprising a sodium-dependent glucose transporter (SGLT2) inhibitor and a method of treating diabetes with a combination comprising a slow release biguanide, e.g., metformin, and a slow release sodium-dependent glucose transporter (SGLT2) inhibitor, and optionally at least one pharmaceutically acceptable excipient and an immediate release layer comprising a sodium-dependent glucose transporter (SGLT2) inhibitor that can provide continuous and non-pulsating therapeutic levels of the biguanide drug to an animal, e.g., human, in need of such treatment over a twelve hour or twenty-four hour period.

In another aspect, the invention also provides a pharmaceutical composition comprising a slow release biguanide, e.g., metformin, a slow release sodium-dependent glucose transporter (SGLT2) inhibitor and optionally at least one pharmaceutically acceptable excipient and a method of treating diabetes with a pharmaceutical composition comprising a slow release biguanide and slow release sodium-dependent glucose transporter (SGLT2) inhibitor and at least one pharmaceutically acceptable excipient.

In yet another aspect, the invention provides a sodium-dependent glucose transporter (SGLT2) inhibitor and a slow release the biguanide, e.g., metformin, wherein the active agents are administered in suboptimal dosages.

In yet another aspect, the invention provides a sodium-dependent glucose transporter (SGLT2) inhibitor and a slow release the biguanide, e.g., metformin, wherein the active agents are administered in amounts and for a sufficient time to produce a synergistic effect.

In yet another aspect, the invention provides pharmaceutical compositions comprising pharmaceutically acceptable salts of the sodium-dependent glucose transporter (SGLT2) inhibitors and the biguanide, e.g., metformin.

In yet another aspect, there is provided a dosage form that affords a steady delivery of a sodium-dependent glucose transporter (SGLT2) inhibitor and a biguanide, e.g., metformin, wherein the peak plasma levels of the biguanide compound is from about 8 to about 12 hours after administration and peak plasma levels of a sodium-dependent glucose transporter (SGLT2) inhibitor from about 1 to about 4 hours after dosing.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates changes in fasting plasma glucose (FPG) (+/−−) SEM) during administration of: 1) a fixed dose combination (FDC) of Dapagliflozin 5 mg and slow release Metformin 500 mg (composition of Example 1, Two Tablets), 2) Glucophage XR 500 mg (2 tablets) and 3) Dapagliflozin 5 mg (composition of Reference Example 1, Two Tablets), all drugs administered once daily orally.

FIG. 2 illustrates changes in hemoglobin A1c (HbA1c) (+/− SEM) during administration of: 1) a fixed dose combination (FDC) of Dapagliflozin 5 mg and slow release Metformin 500 mg (composition of Example 1, Two Tablets), 2) Glucophage XR 500 mg (2 tablets) and 3) Dapagliflozin5 mg (composition of Reference Example 1, Two Tablets), all drugs administered once daily orally.

FIG. 3 illustrates changes in the mean (Δ) in fasting plasma glucose (FPG) (+/−) SEM) during administration of: 1) a fixed dose combination (FDC) of Dapagliflozin 5 mg and slow release Metformin 500 mg (composition of Example 1, Two Tablets), 2) Glucophage XR 500 mg (2 tablets) and 3) Dapagliflozin 5 mg (composition of Reference Example 1, Two Tablets), all drugs administered once daily orally.

FIG. 4 illustrates changes in the mean (A) in hemoglobin A1c (HbA1c) (+/−.SEM) during administration of: 1) a fixed dose combination (FDC) of Dapagliflozin 5 mg and slow release Metformin 500 mg (composition of Example 1, Two Tablets), 2) Glucophage XR 500 mg (2 tablets) and 3) Dapagliflozin 5 mg (composition of Reference Example 1, Two Tablets), all drugs administered once daily orally.

FIG. 5 illustrates the mean plasma concentration of dapagliflozin after administration of single dose of dapagliflozin 5 mg (composition of Reference Example 1) and Slow Release Metformin 500 plus Slow Release Dapagliflozin 2.5 mg plus Immediate Release Dapagliflozin 2.5 mg (composition of Example 10).

FIG. 6 illustrates the mean plasma concentration of dapagliflozin after administration of single dose of Dapagliflozin 5 mg×2 and Slow Release Metformin 850 plus Slow Release Dapagliflozin 10 mg (composition of Example 9).

### DETAILED DESCRIPTION

The terms “a,” “an,” “the,” “at least one,” and “one or more” are used interchangeably. Thus, for example, a composition that comprises “an” element means one element or more than one element.

The term “Active agent or Active agents” refers to various pharmaceutically equivalent isomers, enantiomers, complexes, salts, hydrates, polymorphs, esters and the like of SGLT2 inhibitors, biguanides, sulfonylureas, PPAR agents, α-glycosidase inhibitors, Dual PPAR, Dipeptidyl Peptidase IV inhibitors (DPP IV inhibitors) and Glucagon-like peptide-1 analogs. Non-limiting examples of 1) SGLT2 inhibitors include Dapagliflozin, Remogliflozin, Sergliflozin, Canagliflozin and the like; 2) Non-limiting examples of biguanides include metformin, phenformin, buformin, salts thereof and the like; 3) Non-limiting examples of sulfonylureas include as glipizide, glimiperide, glyburide, glibornuride, glisoxepide, gliclazide acetohexamide, chlorpropamide, tolazamide, tolbutamide and the like; 4) Non-limiting examples of PPAR agents include Troglitazone, Pioglitazone, Rosiglitazone, Ciglitazone, Isaglitazone, Darglitazone, zorglitazone, Englitazone, Balaglitazone, and the like; 5) Non-limiting examples of α-glycosidase inhibitors include acarbose and miglitol, and the like; 6) Non-limiting examples of meglitinides include Repaglinide, Nateglinide, and the like; 7) Non-limiting examples of Dual PPAR agonists include Aleglitazar, Muraglitazar, Tesaglitazar, and the like; 8) Non-limiting examples of Dipeptidyl Peptidase IV inhibitors (DPP IV inhibitors) include Sitagliptin, Vildagliptin, Alogliptin, Saxagliptin, Dutogliptin, Linagliptin and the like; and 9) Non-limiting examples of Glucagon-like peptide-1 analogs include Exenatide, Liraglutide, Albiglutide, Taspoglutide and the like.

The terms “Sodium-dependent glucose transporter SGLT2”, “SGLT2 Inhibitor or SGLT Inhibitors” refers to chemical entities such as Dapagliflozin, Remogliflozin, Sergliflozin, ISIS 388626, JNJ 28431754/TA-7284, BI 10773, BI 44847, and AVE 2268, etc that inhibit Sodium-dependent glucose co-transporters. Non-limiting examples include but are not limited to Dapagliflozin, Remogliflozin, Sergliflozin, ISIS 388626, JNJ 28431754, TA-7284, BI 10773, BI 44847, and AVE 2268 and the like or their pharmaceutically acceptable salts. For Example, the term Dapagliflozin includes salts such as dapagliflozin propylene glycol hydrate, and the like, the term Sergliflozin includes salts such as Sergliflozin etabonate, and the like, and the term Remogliflozin includes salts such as Remogliflozin etabonate, and the like.

The term “Sulfonylurea” refers to drugs such as glipizide, glimiperide, glyburide, glibornuride, glisoxepide, gliclazide acetohexamide, chlorpropamide, tolazamide, and tolbutamide, among others that control or manage non-insulin-dependent diabetes mellitus (NIDDM) by stimulating the release of endogenous insulin from the beta cells of the pancreas.

The term “Thiazolidinediones” includes compounds such as Troglitazone, Pioglitazone, Rosiglitazone, Ciglitazone, Isaglitazone, Darglitazone, zorglitazone, Englitazone, Balaglitazone, and the like.

The term “α-Glycosidase Inhibitors” includes compounds such as α-glucosidase inhibitors, acarbose, miglitol and the like. Additional compounds in this class include acarbose, miglitol, voglibose, emiglitate, and the like.

The term “Meglitinides” refers to a class of drugs that includes Repaglinide, Nateglinide and the like.

The term “diabetes” refers to the diabetes and diabetes related diseases such as type 1 diabetes, type 2 diabetes, hyperglycemia, type 1.5 diabetes, latent autoimmune diabetes (e.g., in adults), maturity onset diabetes, beta-cell apoptosis, hemochromatosis induced diabetes, impaired glucose tolerance, metabolic syndrome X, insulin resistance, cystic fibrosis related diabetes, polycystic ovarian syndrome, gestational diabetes, obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, intermittent claudication (atherosclerosis oblilterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, irritable bowel syndrome, diabetic retinopathy, background retinopathy, preproliferative retinopathy, proliferative retinopathy, macular edema, cataracts, nephropathy, diabetic nephropathy, microalbuminuria, macroalbuminuria, neuropathy, diabetic neuropathy, distal symmetrical sensorimotor polyneuropathy, and diabetic autonomic neuropathy.

The term “co-administration” means administration of the two compounds (e.g., drugs or active agents) to a patient within a period of one day. The term includes separate administration of two medicaments (drugs or active agents) each containing one of the compounds as well as simultaneous administration where the two compounds may be combined in one formulation or administered in two separate formulations.

A “therapeutically effective amount” of a compound is that amount of compound which is sufficient to provide a beneficial effect to the subject to which the compound is administered. For example, a therapeutically effective amount of a biguanide is an amount that can control blood glucose by inhibiting hepatic glucose production.

The term “medicament” means a pharmaceutical composition suitable for administration of the pharmaceutically active compound (drug or active agent) to a patient.

The term “suboptimal dosage” means a dosage that is below the optimal dosage for that compound when used in single-compound therapy.

The term “additive effect” means the effect resulting from the sum of the effects obtained from the individual compounds is equal to the sum of their individual effects in isolation.

The term “synergistic effect” means an effect, which is greater than the additive effect that results from the sum of the effects of the two individual compounds.

The term “treating or treatment” means the management and care of a patient having developed the disease, condition or disorder and includes prophylaxis of the specific disorder or condition, or alleviation of the symptoms associated with a specific disorder or condition or preventing or eliminating said symptoms. Treatment includes the administration of the active compounds to eliminate or control the disease, condition or disorder as well as to alleviate the symptoms or complications associated with the disease, condition or disorder.

The term “prevention of a disease” refers to the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “slow-release” refers to a formulation that is other than an immediate release, e.g., wherein the release of the active ingredient is slow in nature. This includes various terms used interchangeably in the pharmaceutical context such as extended release, delayed release, sustained release, controlled release, timed release, specific release, targeted release etc. These include bilayer formulations wherein at least one active agent is release slowly compared to the other.

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release, etc.

The term “pharmaceutically acceptable derivative” means various pharmaceutically equivalent isomers, enantiomers, complexes, salts, hydrates, polymorphs, esters etc of an SGLT2 inhibitor.

The term “seal coat” refers to a coating that does not contain an active pharmaceutical ingredient and that typically will rapidly disperse or dissolve in water.

“Instructional material” includes a publication, a recording, a diagram, or any other medium of expression that can be used to communicate the usefulness of the composition of the invention for its designated use. The instructional material of the kit of the invention may, for example, be affixed to a container that contains the composition or be shipped together with a container that contains the composition. Alternatively, the instructional material may be shipped separately from the container with the intention that the instructional material and the composition be used cooperatively by the recipient.

Exemplary biguanides include drugs that are useful in controlling or managing diabetes. Non-limiting examples of biguanides include metformin, phenformin or buformin and the like and pharmaceutically acceptable salts, or isomers thereof.

Exemplary sodium-dependent glucose transporter SGLT2 inhibitors include drugs that are useful for controlling or managing diabetes. Non-limiting examples of SLGT2 inhibitors include Dapagliflozin, Remogliflozin, Sergliflozin, ISIS 388626, JNJ 28431754/TA-7284 (Canagliflozin), BI 10773, BI 44847, and AVE 2268 and the like.

Typical combinations include a first active agent present in a slow or controlled release formulation and a second active agent present in an immediate release form. In another combination, a portion of the first active agent is present in a slow or controlled release formulation and a portion is in an immediately release form, and the second active agent is present in an immediate release form. [

An exemplary combination includes a sodium-dependent glucose transporter SGLT2 inhibitor with a slow release metformin.

Another exemplary combination includes dapagliflozin with slow release metformin.

Another exemplary combination includes remogliflozin with slow release Metformin.

Another exemplary combination includes sergliflozin with slow release metformin.

These combinations can produce better than expected therapeutic benefit in the treatment of diabetes and diabetes related diseases.

The invention provides a composition comprising a sodium-dependent glucose transporter SGLT inhibitor in combination with slow release biguanide to treat diabetes and diabetes related diseases and to improve glycemic control in patients in need of treatment.

Further, the invention provides a pharmaceutical composition comprising a sodium-dependent glucose transporter (SGLT2) inhibitor and a slow release biguanide, wherein at least about 85% of the total amount of an at least one active agent is released from the dosage form within about 120 minutes or less. Preferably, at least about 95% of an at least one active agent is released within about 90 minutes when tested in a USP type 1 apparatus, at pH 2.0 in a HCl-0.3M KCl buffer solution.

In another aspect, the active agents can be employed individually, or can be combined in a single formulation, for example as a tablet, capsule, syrup, solution, as well as controlled release formulations. In a preferred embodiment, an immediate release active agent and a slow release active agent are formulated individually and administered in the same manner that each is normally used clinically.

In another aspect, the invention provides an anti-diabetic combination comprising administration of a pharmaceutical composition comprising a slow release biguanide, a slow release sodium-dependent glucose transporter (SGLT2) inhibitor and optionally at least one pharmaceutically acceptable excipient, for the treatment of diabetes and diabetes related diseases. Further, the invention provides a pharmaceutical composition comprising a slow release biguanide, a slow release sodium-dependent glucose transporter (SGLT2) inhibitor and optionally at least one pharmaceutically acceptable excipient, wherein the composition exhibits the following dissolution profile: after about 2 hours 0 to about 25% of the metformin is released; after about 4 hours about 20 to about 45% of the metformin is released; after about 8 hours about 45 to about 90% of the metformin is released, when tested in a USP type 2 apparatus, paddle, at 75 rpms in 900 mL of simulated intestinal fluid, pH 7.5 phosphate buffer and at 37° C. In another embodiment, after about 12 hours at least about 60% of the metformin is released; and after about 16 hours at least about 70% of the metformin is released, when tested in a USP type 2 apparatus, paddle, at 75 rpms in 900 mL of simulated intestinal fluid, pH 7.5 phosphate buffer and at 37° C.

Exemplary sodium-dependent glucose transporter (SGLT2) inhibitors are Dapagliflozin, Remogliflozin, Sergliflozin, ISIS 388626, JNJ 28431754/TA-7284, BI 10773, BI 44847, and AVE 2268 or a pharmaceutically acceptable salt thereof.

In another aspect, the invention provides an anti-diabetic combination comprising administration of a pharmaceutical composition comprising a slow release biguanide and a slow release sodium-dependent glucose transporter (SGLT2) inhibitor and optionally at least one pharmaceutically acceptable excipient, for the treatment of diabetes and diabetes related diseases. Further, the invention provides a method of treating diabetes comprising administration of a pharmaceutical composition comprising a slow release biguanide and a slow release sodium-dependent glucose transporter (SGLT2) inhibitor and optionally at least one pharmaceutically acceptable excipient, which exhibits the following dissolution profile: after about 2 hours 0 to about 25% of the DPP IV inhibitor is released; after about 4 hours about 20 to about 55% of the SGLT2 inhibitor is released; after about 8 hours about 45 to about 90% of the SGLT2 inhibitor is released; when tested in a USP type 2 apparatus, paddle, at 75 rpms in 900 ml of simulated intestinal fluid, pH 7.5 phosphate buffer and at 37° C. In another embodiment, after about 12 hours not less than 80% of the SGLT2 inhibitor is released; and after about 16 hours not less than 90% of the SGLT2 inhibitor is released; when tested in a USP type 2 apparatus, paddle, at 75 rpms in 900 ml of simulated intestinal fluid, pH 7.5 phosphate buffer and at 37° C.

In another embodiment, the invention provides an anti-diabetic combination comprising administration of a pharmaceutical composition comprising a slow release sodium-dependent glucose transporter (SGLT2) inhibitor at least one pharmaceutically acceptable excipient, and an immediate release layer comprising biguanide, for the treatment of diabetes and diabetes related diseases, which exhibits the following dissolution profile: after about 2 hours 0 to about 25% of the sodium-dependent glucose transporter (SGLT2) inhibitor is released; after about 4 hours about 20 to about 55% of the sodium-dependent glucose transporter (SGLT2) inhibitor is released; after about 8 hours about 45 to about 90% of the sodium-dependent glucose transporter (SGLT2) inhibitor is released; when tested in a USP type 2 apparatus, paddle, at 75 rpms in 900 ml of simulated intestinal fluid, pH 7.5 phosphate buffer and at 37° C. In another embodiment, after about 12 hours at least about 80% of the sodium-dependent glucose transporter (SGLT2) inhibitor is released; and after about 16 hours at least about 90% of the sodium-dependent glucose transporter (SGLT2) inhibitor is released, when tested in a USP type 2 apparatus, paddle, at 75 rpms in 900 ml of simulated intestinal fluid, pH 7.5 phosphate buffer and at 37° C.

In another aspect, the invention further provides an anti-diabetic combination comprising administration of a pharmaceutical composition comprising a slow release sodium-dependent glucose transporter (SGLT2) inhibitor at least one pharmaceutically acceptable excipient, and an immediate release biguanide, for the treatment of diabetes and diabetes related diseases. Further, the invention provides a pharmaceutical composition comprising administration of a pharmaceutical composition comprising a slow release sodium-dependent glucose transporter (SGLT2) inhibitor at least one pharmaceutically acceptable excipient, and an immediate release biguanide, that exhibits the following dissolution profile: after about 2 hours at least about 70% of the metformin is released, when tested in a USP type 2 apparatus, paddle, at 75 rpms in 900 ml of simulated intestinal fluid, pH 7.5 phosphate buffer and at 37° C.

An exemplary core includes an osmotic tablet core with or without a gelling or swelling polymer. The tablet core includes an active agent and can include at least one pharmaceutically acceptable excipient. This active agent is preferably delivered in a controlled release manner (slow release), e.g., from a tablet core. An example of an active agent in a slow release core includes the biguanide, optionally a binding agent and an optional absorption enhancer. The tablet core is preferably coated with a polymeric coating to form a membrane around the tablet and drilled to create one passageway on each side of the membrane. A preferred biguanide is metformin or a pharmaceutically acceptable salt thereof.

The compositions can optionally include an absorption enhancer, which can be any type of absorption enhancer commonly known in the art such as a fatty acid, a surfactant (anionic, cationic, amphoteric), a chelating agent, a bile salt and the like or mixture thereof. Non-limiting examples of absorption enhancers include lecithin, fatty acids such as capric acid, oleic acid, monoglycerides thereof and the like, surfactants such as sodium lauryl sulfate, sodium taurocholate and polysorbate 80 and the like, chelating agents such as citric acid, phytic acid, ethylenediamine tetra acetic acid (EDTA) and ethylene glycol-bis(β-amino ethyl ether)-N,N,N,N-tetra acetic acid (EGTA) and the like. The core may include from 0 to about 20 weight % absorption enhancer based on the total weight of the core and preferably about 2% to about 10 weight % of the total weight of the core.

The core of is can be formed by granulating an active agent with a binding agent and compressing the granules with a lubricant and an absorption enhancer into a tablet. The core may also be formed either by dry granulating the core ingredients into a mixture and passing the mixture through a roller compactor and compressing the granules, with a lubricant, into tablets or by direct compression. The cores can also be prepared using other commonly known granulation procedures that are known in the art. For example other excipients such as lubricants, pigments or dyes known in the art may also be employed in the formulation of the subject invention.

A membrane or sustained release coating may be used to coat the core. Non-limiting examples of materials useful in forming a membrane or slow release coating include ethylcellulose, cellulose esters, cellulose diesters, cellulose triesters, cellulose ethers, cellulose ester-ether, cellulose acrylate, cellulose diacrylate, cellulose triacrylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008. A preferred coating material is cellulose acetate, having an acetyl content of 39.3 to 40.3%, which is commercially available from Eastman Fine Chemicals.

Optionally a flux-enhancing agent can be included in the membrane or slow release coating. The flux-enhancing agent can increase the volume of fluid imbibed into the core to enable the dosage form to dispense substantially all of the biguanide through the passage or the porous membrane. The flux-enhancing agent can be a water-soluble material or an enteric material. Non-limiting examples of pore forming flux enhancers include sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F127, LUTROL F108 which are commercially available from BASF) or mixture thereof. A preferred flux-enhancing agent is PEG 400.

The flux enhancer may also be a water-soluble drug such as metformin or a pharmaceutically acceptable salt, or the flux enhancer may be a drug that is soluble under intestinal conditions. If the flux enhancer is a drug, the present pharmaceutical composition has an added advantage of providing an immediate release of the drug selected as the flux enhancer. The flux-enhancing agent can dissolve or leach from the membrane or sustained release coating to form channels in the membrane or sustained release coating which enables fluid to enter the core and dissolve the active ingredient. Preferably, the flux-enhancing agent is from 0 to about 40% of the total weight of the coating, most preferably from about 2% to about 20 weight % of the total weight of the coating.

Excipients such as plasticizers may be used for preparing the membrane or slow release coating. Non-limiting examples of plasticizers include adipates, azelates, enzoates, citrates such as triethyl citrate, tri-n-butyl citrate, acetyl tri-n-butyl citrate, acetyltributylcitrate, acetyltriethylcitrate and the like, stearates, isoebucates, sebacates, and plasticizers described in the Encyclopedia of Polymer Science and Technology, Vol. 10 (1969), published by John Wiley & Sons. Preferred plasticizers include triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and the like. The exact amount of plasticizer used depends on the type of plasticizer. Typically, the plasticizer can be from 0 to about 25 weight % are used, and preferably about 2 to about 15 weight % based upon the total weight of the membrane or sustained release coating.

Generally, the membrane or slow release coating can comprise from about 1 to about 10 weight % and preferably about 2 to about 5 weight % based upon the total weight of the core and coating.

The membrane or sustained release coating surrounding the core further comprises a passage that can allow for controlled release of the drug from the core in a preferred embodiment. The term “passage” includes an aperture, orifice, bore, hole, weakened area or a credible element such as a gelatin plug that erodes to form an osmotic passage for the release of the biguanide from the dosage form. Exemplary passages are well known and described, e.g., in U.S. Pat. Nos. 3,845,770; 3,916,899; 4,034,758; 4,077,407; 4,783,337 and 5,071,607.

The present invention provides a combination that includes a sodium-dependent glucose transporter (SGLT2) inhibitor that is independent of the second active agent used in the combination. In one example, the SGLT2 inhibitor is formulated to provide an immediate release of the inhibitor. In another example the sodium-dependent glucose transporter (SGLT2) inhibitor can be applied in the form of a layer to a controlled or slow released core comprising the a second active agent as a layer using a binder and other conventional pharmaceutical excipients such as absorption enhancers, surfactants, plasticizers, antifoaming agents and combinations disclosed above. An absorption enhancer may be present in a sodium-dependent glucose transporter (SGLT2) inhibitor layer in an amount up to about 10 to about 30 weight % based on the total weight of the layer. A binding agent may be present in an amount up to about 5 to about 150 weight % based on the weight of a sodium-dependent glucose transporter (SGLT2) inhibitor.

The present invention also provides a pharmaceutical composition comprising a sodium-dependent glucose transporter SGLT2 inhibitor and a biguanide both the active agents are formulated as bilayer formulation. The bilayer formulation includes the dosage forms wherein at least one of the active agents is in slow release form.

The immediate release formulation can be incorporated into a single dosage form by coating a layer containing the active ingredient onto the membrane or slow release coating of the dosage form using conventional methods. Alternatively, the second active ingredient may also be incorporated by any pharmaceutically acceptable method into a single dosage form with the first active ingredient. The incorporation of the second active ingredient may be performed, among others, by commonly used processes including drug layering, lamination, dry compression, deposition and printing.

When an active agent, such as a sodium-dependent glucose transporter (SGLT2) inhibitor, is coated onto a membrane or slow release coating of an osmotic tablet core, the active agent, such as a sodium-dependent glucose transporter (SGLT2) inhibitor, coating can be applied from a coating solution or suspension that employs an aqueous solvent, an organic solvent or a mixture of an aqueous and an organic solvent. Exemplary organic solvents include acetone, isopropyl alcohol, methanol, ethanol and the like. When a mixture of aqueous and organic solvents is employed, the ratio of water to organic solvent should be in the range from 98:2 to 2:98, preferably 50:50 to 2:98, more preferably 30:70 to 20:80 and most preferably from about 25:75 to about 20:80. When mixed solvent systems are employed, the amount of binder required for coating the active agent, such as a sodium-dependent glucose transporter (SGLT2) inhibitor, onto the membrane or a slow release coating can be reduced. For example, successful coatings have been obtained from a mixed solvent system where the weight ratio of binder to an active agent, such as a sodium-dependent glucose transporter (SGLT2) inhibitor, is 1:9 to 1:11. Although acceptable coatings can be obtained when the active agent coat is applied directly to the membrane or slow release coating, a preferred method is to first coat the membrane or slow release coating with a seal coat prior to the application of the active agent, such as a sodium-dependent glucose transporter (SGLT2) inhibitor coating.

In one example, the active agent, such as a sodium-dependent glucose transporter (SGLT2) inhibitor, coating solution or suspension can include a surfactant and a pore forming agent such as sodium chloride, potassium chloride, sucrose, sorbitol, mannitol, polyethylene glycols (PEG), propylene glycol, hydroxypropyl cellulose, hydroxypropyl methylcellulose, hydroxypropyl methylcellulose phthalate, cellulose acetate phthalate, polyvinyl alcohols, methacrylic acid copolymers, poloxamers. In another example, the pharmaceutical composition of the present invention may also include an effective immediate release amount of an active agent, such as biguanide. The effective immediate release amount of an active agent, such as biguanide, can be coated onto the membrane or slow release coating of the dosage form or it may be incorporated into the membrane or slow release coating.

In addition, various diluents, excipients, lubricants, dyes, pigments, dispersants, etc., which are disclosed in Remington's Pharmaceutical Sciences (1995), may be used to optimize the above listed formulations of the subject invention.

Sodium-dependent glucose transporter (SGLT2) inhibitors such as dapagliflozin can be administered at levels of about 0.5, 1.0, 2.5, 5, 10, 20, 50, 100 or 200 milligrams per day, Biguanides, such as metformin are commonly administered in dosage forms containing about 500 mg, 750 mg, 850 mg, and 1000 mg. Sodium-dependent glucose transporter (SGLT2) inhibitors, for example sitagliptin, are commonly administered in dosage forms containing about 2.5 mg, 5 mg, 10 mg 25 mg 50 mg and 100 mg. The present invention is intended to encompass the above listed therapeutic combinations, in each of each possible combination of compounds and their respective dosage amounts.

The use of a binding agent in the core is optional. Exemplary binding agents include conventional pharmaceutically acceptable binders known in the art such as polyvinyl pyrrolidone, hydroxypropyl cellulose, hydroxyethyl cellulose, hydroxypropyl methylcellulose, ethylcellulose, polymethacrylate, polyvinyl alcohol, waxes and the like or mixtures thereof. Preferred binding agents are water soluble materials such as polyvinyl pyrrolidone having a weight average molecular weight of 25,000 to 3,000,000. The binding agent may comprise approximately about 0 to about 40 weight % of the total weight of the core and preferably about 3% to about 15 weight % based on the total weight of the core.

Exemplary hydrophilic polymers include, but are not limited, to hydroxypropyl-methylcellulose, hydroxypropylcellulose, sodium carboxymethylcellulose, carboxymethyl-cellulose calcium, ammonium alginate, sodium alginate, potassium alginate, calcium alginate, propylene glycol alginate, alginic acid, polyvinyl alcohol, povidone, carbomer, potassium pectate, potassium pectinate, and the like or mixtures thereof.

Exemplary extended release materials for use in the inner solid particulate phase or the outer solid continuous phase include one or more hydrophilic polymers, one or more hydrophobic polymers, or one or more other type hydrophobic materials, such as, for example, one or more waxes, fatty alcohols or fatty acid esters. The extended release material in the inner solid particulate phase may be the same as or different from an extended release material present in the outer solid continuous phase.

Exemplary hydrophobic polymers include, but are not limited, to ethyl cellulose, hydroxyethylcellulose, amino methacrylate copolymer (Eudragit RL™. or Eudragit RS™), methacrylic acid copolymers (Eudragit L™. or Eudragit S™), methacrylic acid-acrylic acid ethyl ester copolymer (Eudragit L 100-5™), methacrylic acid esters neutral copolymer (Eudragit NE 30D.™), dimethylaminoethylmethacrylate-methacrylic acid esters copolymer (Eudragit E 100™), vinyl methyl ether/malefic anhydride copolymers, their salts and esters (Gantrez™) and the like or mixtures thereof.

Additional hydrophobic materials which can be employed in the inner solid particulate phase or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, and the like or mixtures thereof.

Exemplary gelling or swelling polymers include polymers that gel, swell or expand in the presence of water or biological fluids. Non-limiting examples of gelling or swelling polymers are high molecular weight hydroxypropyl methylcellulose. Examples of hydroxypropyl methylcelluloses that are commercially available are METHOCEL E (USP type 2910), METHOCEL F (USP type 2906), METHOCEL J (USP type 15 1828), METHOCEL K (USP type 2201), and METHOCEL 310 Series, products of The Dow Chemical Company. Other products such as METHOCEL™, K100M, which is commercially available from Dow Chemical) and high molecular weight polyethylene oxides (such as POLYOX™ WSR 301, WSR 303 or WSR COAGULANT) are also used. Other gelling or swelling polymers are described in U.S. Pat. No. 4,522,625. In additional examples, the invention uses the poly(ethylene oxide) to provide superior gastric retention. Poly(ethylene oxide), also referred to herein as “polyethylene oxide” is a linear polymer of un-substituted ethylene oxide. Poly(ethylene oxide)polymers having viscosity-average molecular weights of about 100,000 Daltons or more can be used in accordance with this invention.

The dosage of each active agent (compound) that is administered can be determined the attending physician who would consider the severity of the disease, the frequency of administration, the particular agents and combinations utilized, and other factors routinely considered in a diabetic practice. Typically, sodium-dependent glucose transporter (SGLT2) inhibitors will normally be administered at doses from about 0.5 mg to about 500 mg per day, and more preferably from about 2 mg to about 100 mg per day. A preferred sodium-dependent glucose transporter (SGLT2) inhibitor is dapagliflozin, and it typically will be employed at doses from about 0.5 mg to about 100 mg per day. Slow release active agents such as metformin hydrochloride can be administered at doses of about 300 mg to about 2000 mg per day. Metformin hydrochloride is commercially available in tablets that contain 500 mg, 750 mg and 1000 mg of active agent. The number and frequency of the dosages administered depends on the nature of the disease and the conditions of the patients but can be given up to two times a day or more.

The invention provides compositions of anti-diabetic combinations, for example, a sodium-dependent glucose transporter (SGLT2) inhibitor and a slow release biguanide, and a method of treating diabetes and controlling glycemic conditions including administering to a patient in need of such treatment an effective amount of a sodium-dependent glucose transporter (SGLT2) inhibitor and a slow release biguanide. When a sodium-dependent glucose transporter (SGLT2) inhibitor and a slow release biguanide are formulated together, the compositions can have from about 0.5 and to about 1000 mg of weight of a sodium-dependent glucose transporter (SGLT2) inhibitor and about 100 to about 2000 mg of biguanide. For example, a typical two-way composition can include 2.5 or 5.0 mg of dapagliflozin and 750-850 mg of metformin. As a second example, a typical two-way composition can include 10 mg of sergliflozin and 750-850 mg of Metformin. As a third example, a typical two-way composition can include 5 mg of remogliflozin and 750-850 mg of metformin. The compositions may contain common excipients and carriers such as starch, sucrose, polymers, talc, gelatin, methylcellulose, magnesium stearate and the like or mixtures thereof. The compositions will typically be prepared for oral administration, for instance as tablets or capsules, but also may be in the form of aqueous suspensions or solutions, suppositories, slow release forms, for example employing an osmotic pump, skin patch, or the like.

The disclosed compositions include a kit comprising composition comprising of a sodium-dependent glucose transporter (SGLT2) inhibitor and a biguanide, wherein one of the active agents is in slow release form and instructional material that describes administering the composition to a subject. This should be construed to include other embodiments of kits that are known to those skilled in the art, such as a kit comprising a (preferably sterile) solvent for dissolving or suspending the composition prior to administering the composition to subject. Preferably, the subject is a human.

Exemplary compositions of a pharmaceutical composition comprising a sodium-dependent glucose transporter (SGLT2) inhibitor and a biguanide, using dapagliflozin can have the composition disclosed in Table 1:

TABLE 1

| | Range percent | Preferred Range % |
|---|---|---|
| First Active Ingredient | | |
| Drug | 50-98% | 75-95% |
| Binder | 0.1-40% | 3-15% |
| Absorption Enhancer | 0-20% | 2-10% |
| Lubricant | 0-5% | 0.5-1% |
| Coating | | |
| Polymer | 50-99% | 75-95% |
| Flux Enhancer | 0-40% | 2-20% |
| Plasticizer | 0-25% | 2-15% |
| Second Active Ingredient | | |
| Drug | 0.1-20% | 1-10% |
| Binder | 0.1-20% | 1-15% |
| Surfactant | 0-20% | 0.1-15% |
| Pore Former | 0-25% | 0.1-15% |
| Polymer (Optional) | 0-30% | 0.1-20% |

The invention is now described with reference to the following Examples. Without further description, it is believed that one of ordinary skill in the art can, using the preceding description and the following illustrative examples, make and utilize the disclosed compositions. The following working examples therefore, are provided for the purpose of illustration only and specifically point out the preferred embodiments, and are not to be construed as limiting in any way the remainder of the disclosure. Therefore, the examples should be construed to encompass any and all variations which become evident as a result of the teaching provided herein.

### Example 1

### Preparation of Metformin Hydrochloride/Dapagliflozin Composition

A slow-release tablet containing 500 mg of metformin HCl and 5 mg dapagliflozin is prepared using a three step process: 1) Granulation, 2) Tableting and 3) Membrane coating process. An optional seal coating may be applied to the core tablet. The specific steps are described below.

Granulation: Metformin hydrochloride was screened using a size reduction and screening equipment (Comil screener) and was further fluidized using a commercially available powder coater granulator (Glatt 60). It was sprayed with a povidone solution prepared in a steel tank using water as the solvent. The spraying was carried at about 2.5 bar pressure by varying the pump rate from 0-15 minutes for a target of 500 g/min. to achieve a target of about 1200 g/min. in the final phase. Granules were dried until an LOD of less than 2% and passed through a screener (Comil 1143/75).

Tableting: The coated metformin hydrochloride was mixed with sodium lauryl sulfate in a blender (Slant-Cone: 30 minutes). Magnesium stearate was screened and blended with the metformin hydrochloride-sodium lauryl sulfate mixture. The homogenized mixture was compressed into tablets using standard procedures. The metformin hydrochloride core tablets weighted from 650 mg to 800 mg with a frigidity of less than 1%.

Seal coating: Seal coating of the metformin core tablets was accomplished by spraying (O'Hara Lab Coat Pan Coater) a solution of either Opadry coating material. The spraying was conducted at a temperature of 46-47° C., atomization pressure of 40-60 psi at a spray rate of 180 grams per minute/three guns. The pan speed was at 4-8 rpm and air volume of 1000+/−100. The seal coated metformin hydrochloride had a theoretical coating of 2.5-5.0 weight %.

Membrane coating: Cellulose acetate was mixed with acetone to prepare a clear solution. Polyethylene glycol 400 was added this mixture and triacetin was added to the resulting solution. The seal coated metformin hydrochloride tablets were fluidized using a Glatt coater. The cellulose acetate solution was sprayed onto the fluidized seal coated metformin hydrochloride tablets at an atomization pressure of 2.5 bars, using an air volume of 1700 CFM, at spraying rate of about 450 g/ml to achieve coating target of 1.3 weight %. The membrane coated tablets were dried sequentially at temperature of 21° C. and 40° C. An orifice was made on the membrane coated tablets using laser with an average diameter of 0.4 to 0.5 mm with micrometer ranging from 6 to 7 mm. Laser was operated with pulse width of 165+/−65 and a pulse delay of around 340+/−100 respectively.

Manufacturing process of dapagliflozin coating; The above prepared membrane coated metformin hydrochloride tablets were further seal coated with Opadry Clear (YS-1-7006) solution using standard coater such as an O'Hara pan coater, tip set at 4″ at a spray rate of 25 mL/gun/min, exhaust temperature of around 45° C., an atomization pressure from 10-35 psi, at a pan speed of 5-8 rpm, using airflow 350CFM. The dapagliflozin coating solution was prepared carefully and slowly by dissolving Lutrol F-68 in water. Similarly the povidone K-30 in water solution was prepared separately and was mixed with spray dried lactose monohydrate. Following the addition of lactose, dapagliflozin was first dispersed in the above prepared Lutrol solution with constant stirring and finally sodium starch glycolate was added into the coating solution. The dapagliflozin coating was applied to the seal coated 500 mg metformin hydrochloride membrane coated tablets using the above mentioned coater at identical conditions. Over this, 5 mg dapagliflozin coated seal coated 500 mg metformin hydrochloride membrane coated tablets, color coating was done using similar coater and identical conditions mentioned above.

Finally, color coated tablets were dried and polished using Cindrella wax and the finished final tablets were packaged in a HDPE bottle with a suitable desiccant and subjected appropriate stability and clinical studies.

Table 2 illustrates a representative example of a pharmaceutical composition of a slow release composition having a biguanide and an SGLT2 inhibitor. The pharmaceutical composition used was 500 mg metformin hydrochloride and 5 mg of dapagliflozin.

TABLE 2

| | Amount mg/tablet |
|---|---|
| First Active Ingredient | |
| Metformin HCl | 500.0 |
| Povidone K 301 USP | 30.0 |
| Sodium Lauryl Sulfate | 26.0 |
| Magnesium Stearate | 2.8 |
| Seal Coat | |
| Opadry Clear (YS 1-7006) | 24.0 |
| Semi Permeable Coat | |
| Cellulose Acetate (398-10) NF | 7.6 |
| Triacetin | 0.5 |
| PEG 400 | 0.9 |
| Seal Coat | |
| Opadry Clear (YS 1-7006) | 5.0 |
| Second Active Ingredient | |
| Dapagliflozin | 5.0 |
| Povidone K 30 USP | 1.0 |
| Lactose Monohydrate | 5.0 |
| Sodium starch Glycolate | 2.5 |
| Poloxamer 188 | 1.0 |
| HPMC | 1.0 |
| PEG 8000 | 0.1 |
| Titanium Dioxide | 0.1 |
| Wax | 0.1 |

The dosage forms prepared above exhibit the dissolution profile illustrate in Table 3 when tested in a USP Type 2 apparatus at 75 rpm in 900 ml of simulated intestinal fluid (pH 7.5 phosphate buffer) and at 37° C.

TABLE 3
Dissolution Profile

| | | |
|---|---|---|
| Time hours | Percent Release | Range |

Biguanide

| | | |
|---|---|---|
| 2 | 0-25% | 0-15% |
| 4 | 10-45% | 20-40% |
| 8 | 30-90% | 45-90% |
| 12 | >50% | >60% |
| 16 | >60% | >60% |
| 20 | >70% | >70% |

Dapagliflozin

| | | |
|---|---|---|
| 1 | >30% | >30% |

The selection of the excipients for use in the immediate release layer of the dosage form can greatly affect the release characteristics, potency and stability of the sodium-dependent glucose transporter (SGLT2) inhibitor. Therefore, in an alternate example, the composition of the sodium-dependent glucose transporter (SGLT2) inhibitor component of the present invention should be selected so that at least about 50%, preferably at least about 80% and most preferably at least about 95% of the sodium-dependent glucose transporter (SGLT2) inhibitor is released from the dosage form within 120 minutes, preferably within 90 minutes and most preferably within 60 minutes when tested according to the United States Pharmacopeia (USP) 26, with Apparatus 1 at 100 rpm, 37′C and 900 ml of 0.3 M KCl—HCl Buffer, pH 2.0.

The excipients for use in the sodium-dependent glucose transporter SGLT2 inhibitor layer of the dosage form are selected so that the total sodium-dependent glucose transporter SGLT2 inhibitor related compounds or impurities in the final dosage form are less than about 0.6 weight %, preferably less than about 0.5 weight % and most preferably less than about 0.25 weight % and each individual sodium-dependent glucose transporter SGLT2 inhibitor related compound or impurity in the final dosage form is less than about 0.25%, preferably less than about 0.2 weight % and most preferably less than about 0.1 weight %. The sodium-dependent glucose transporter (SGLT2) inhibitor related compounds or impurities in the final dosage form are determined by High Performance Liquid Chromatography (HPLC) using a YMC-ODS-AQ, 5 μm, 120 Å, 4.6.times.250 mm or equivalent column, a 0.1 M ammonium acetate buffer: acetonitrile: glacial acetic acid (25:25:1) mobile phase, about a 40. μL injection volume, 0.7 mL/min flow rate, 25° C. column temperature and 269 nm wavelength for the UV detector.

### Example 2

A pharmaceutical composition comprising Dapagliflozin 10 mg and slow release metformin hydrochloride 1000 mg was manufactured as described in Example 1.

Table 4 shows the representative example of a pharmaceutical composition of a slow release composition having a biguanide and a sodium-dependent glucose transporter (SGLT2) inhibitor. The pharmaceutical composition used was 1000 mg metformin hydrochloride and 10 mg of Dapagliflozin.

TABLE 4

| | Amount mg/tablet |
|---|---|
| First Active Ingredient | |
| Metformin HCl | 1000.0 |
| Povidone K 301 USP | 78.0 |
| Sodium Lauryl Sulfate | 51.7 |
| Magnesium Stearate | 5.7 |
| Seal Coat | |
| Opadry Clear (YS 1-7006) | 47.0 |
| Semi Permeable Coat | |
| Cellulose Acetate (398-10) NF | 15.5 |
| Triacetin | 0.9 |
| PEG 400 | 1.8 |
| Seal coat | |
| Opadry Clear (YS 1-7006) | 9.0 |
| Second Active Ingredient | |
| Dapagliflozin | 10.0 |
| Povidone K 30 USP | 1.0 |
| Lactose Monohydrate | 10.0 |
| Sodium starch Glycolate | 5.0 |
| Poloxamer 188 | 2.0 |
| HPMC | 2.0 |
| PEG 8000 | 0.2 |
| Titanium Dioxide | 0.2 |
| Wax | 0.2 |

### Example 3

Alternatively, pharmaceutical compositions comprising slow release biguanide and a sodium-dependent glucose transporter (SGLT2) inhibitor (For Example; Dapagliflozin and Remogliflozin) were prepared as a bilayer tablet using standard methods known in the art as exemplified below in Tables 5 and 6:

TABLE 5

| | Amount |
|---|---|
| Layer 1: | |
| Metformin HCl | 500 mg |
| Microcrystalline cellulose | 10-25% |
| Polyvinyl alcohol | 3-5% |
| Ethylcellulose (5-20 cp) | 10-20% |
| Hydroxyethyl cellulose | 5-15% |
| Colloidal silicon dioxide | 2-5% |
| Sodium stearyl fumarate | 1-2% |
| Layer 2: | |
| Dapagliflozin | 250 mg |
| Microcrystalline cellulose | 5-20% |
| Povidone | 10-15% |
| Crosscarmellose sodium | 5-10% |
| Magnesium stearate | 0.5-2% |

TABLE 6

| | Amount |
|---|---|
| Layer 1: | |
| Metformin HCl | 500 MG |
| Microcrystalline cellulose | 10-25% |
| Polyvinyl alcohol | 3-5% |
| Ethylcellulose (5-20 cp) | 10-20% |
| Hydroxyethyl cellulose | 5-15% |
| Colloidal silicon dioxide | 2-5% |
| Sodium stearyl fumarate | 1-2% |
| Layer 2: | |
| Remogliflozin | 100 mg |
| Microcrystalline cellulose | 5-20% |
| Povidone | 10-15% |
| Crosscarmellose sodium | 5-10% |
| Magnesium stearate | 0.5-2% |

### Manufacturing Process:

Preparation of Layer 1: Metformin Hydrochloride, microcrystalline cellulose and colloidal silicon dioxide were granulated with polyvinyl alcohol and dried. The dried granules are mixed with Ethylcellulose and Hydroxyethylcellulose and lubricated with Sodium stearyl fumarate.

Preparation of Layer 2: Dapagliflozin, mixed with microcrystalline cellulose was granulated with povidone. Granules are dried and mixed with Crosscarmellose sodium and finally lubricated with Magnesium stearate.

Compression: Layer 1 and Layer 2 are loaded into the hopper of Bilayer rotary compression machine and compressed with a desired hardness.

The other pharmaceutical compositions comprising a sodium-dependent glucose transporter (SGLT2) inhibitor and a biguanide, wherein one of the active agents is in slow release form were prepared using standard methods known in the art. Examples of 1) Sodium-dependent glucose transporter (SGLT2) inhibitors include Dapagliflozin, Remogliflozin, Sergliflozin, ISIS 388626, JNJ 28431754/TA-7284, BI 10773, BI 44847, and AVE 2268, 2) biguanides include metformin, phenformin, buformin, etc.

### Example 4

As additional examples, pharmaceutical dosage forms comprising a slow release metformin and a sodium-dependent glucose transporter (SGLT2) inhibitor was prepared according to composition outlined below in Table 7 and Table 8

TABLE 7

| | mg/tablet |
|---|---|
| First Active Ingredient | |
| Metformin Hydrochloride | 500 |
| Ethylcellulose | 25 |
| Methocel Premium K 100 | 350 |
| Methocel E3 Premium | 10 |
| Microcrystalline Cellulose | 100 |
| Magnesium Stearate | 7 |
| Ethanol* | 50 |
| Second Active Ingredient | |
| Dapagliflozin | 5.0 |
| Povidone K 30 USP | 1.0 |
| Lactose Monohydrate | 5.0 |
| Sodium starch Glycolate | 2.5 |
| Poloxamer 188 | 1.0 |
| HPMC | 1.0 |
| PEG 8000 | 0.1 |
| Titanium Dioxide | 0.1 |
| Wax | 0.1 |

*Removed during the processing.

### Example 5

TABLE 8

| | mg/tablet |
|---|---|
| First Active Ingredient | |
| Metformin Hydrochloride | 502 |
| Methylcellulose | 50 |
| Methocel Premium K 100 | 360 |
| Methocel E3 Premium | 10 |
| Microcrystalline Cellulose | 102 |
| Magnesium Stearate | 10 |
| Water* | 50 |
| Second Active Ingredient | |
| Dapagliflozin | 5.0 |
| Povidone K 30 USP | 1.0 |
| Lactose Monohydrate | 5.0 |
| Sodium starch Glycolate | 2.5 |
| Poloxamer 188 | 1.0 |
| HPMC | 1.0 |
| PEG 8000 | 0.1 |
| Titanium Dioxide | 0.1 |
| Wax | 0.1 |

Manufacturing Process: The metformin hydrochloride-ethylcellulose granules were prepared by carefully and slowly adding a solution of ethylcellulose in ethanol solvent. The process was carried out in a mixer to prepare uniform granules. The granules were dried at 65′C and passed through a screener (8-10 mm). The granules were blended in a blender with Methocel Premium K10, Methocel Premium E3 and Microcrystalline Cellulose in a mixer for about an hour. The finely blended mixture was further mixed with Magnesium Stearate and compressed into tablets. Optionally the tablets were coated with Opadry Clear (YS-1-7006). The slow release metformin hydrochloride tablets were coated with a layer of Dapagliflozin

Manufacturing process of dapagliflozin coating; The above prepared metformin hydrochloride tablets were further seal coated with Opadry Clear (YS-1-7006) solution using standard coater such as an O'Hara pan coater, tip set at 4″ at a spray rate of 25 mL/gun/min, exhaust temperature of around 45° C., an atomization pressure from 10-35 psi, at a pan speed of 5-8 rpm, using airflow 350CFM. The dapagliflozin coating solution was prepared carefully and slowly by dissolving Lutrol F-68 in water. Similarly the povidone K-30 in water solution was prepared separately and was mixed with spray dried lactose monohydrate. Following the addition of lactose, dapagliflozin was first dispersed in the above prepared Lutrol solution with constant stirring and finally sodium starch glycolate was added into the coating solution. The dapagliflozin coating was applied to the seal coated 500 mg metformin hydrochloride membrane coated tablets using the above mentioned coater at identical conditions. Over this, 5 mg dapagliflozin coated seal coated 500 mg metformin hydrochloride membrane coated tablets, color coating was done using similar coater and identical conditions mentioned above.

The in vitro dissolution profile of Example 4 and 5 are shown in Table 9;

TABLE 9

| Time Hours | Example 4 | Example 5 |
|---|---|---|
| 0 | 0 | 0 |
| 1 | 37.5 | 4.2 |
| 2 | 51.2 | 49.2 |
| 3 | 67.7 | 65.4 |
| 4 | 77.2 | 75.3 |
| 5 | 85.1 | 82.4 |
| 6 | 91.3 | 86.2 |
| 7 | 95.1 | 89.1 |
| 8 | 97 | 90.1 |

### Example 6

In yet another example, a pharmaceutical dosage form comprising a slow release metformin and a sodium-dependent glucose transporter (SGLT2) inhibitor was prepared according to composition below in Table 10

TABLE 10

| | mg/tablet |
|---|---|
| First Active Ingredient | |
| Metformin Hydrochloride | 500 |
| Polyethylene Oxide | 425 |
| Methocel E5 Premium | 55 |
| Microcrystalline Cellulose | 10 |
| Methocel E3 Premium | 9 |
| Magnesium Stearate | 1 |
| Second Active Ingredient | |
| Dapagliflozin | 5 |
| Povidone K 30 USP | 1 |
| Lactose Monohydrate | 5 |
| Sodium starch Glycolate | 2.5 |
| Poloxamer 188 | 1 |
| HPMC | 1 |
| PEG 8000 | 0.1 |
| Titanium Dioxide | 0.1 |
| Wax | 0.1 |

### Manufacturing Process:

Metformin Hydrochloride, Polyethylene Oxide, Methocel E5 Premium, Microcrystalline Cellulose, Methocel E3 Premium and Magnesium Stearate were formulated by dry blending a granulation of composition according to Table 8. The finely blended granules were compressed into tablets using table press (Fred Carver, Inc., Ind.).

### Example 7

In yet another example, a pharmaceutical dosage form comprising a slow release metformin and a sodium-dependent glucose transporter (SGLT2) inhibitor was prepared according to composition in Table No 11:

TABLE 11

| | mg/tablet |
|---|---|
| First Active Ingredient | |
| Metformin Hydrochloride | 500 |
| Glyceryl Behanate | 9.5 |
| Polyvinyl Alcohol | 11.9 |
| Silicon Dioxide | 10.3 |
| Coating | |
| Ethylellulose | 25.5 |
| Povidone K 30 USP | 9.5 |
| Dibutyl Sebacate | 9.6 |
| Magnesium Stearate (Optional) | 1.5 |
| Second Active Ingredient | |
| Dapagliflozin | 5 |
| Povidone K 30 USP | 1 |
| Lactose Monohydrate | 5 |
| Sodium starch Glycolate | 2.5 |
| Poloxamer 188 | 1 |
| HPMC | 1 |
| PEG 8000 | 0.1 |
| Titanium Dioxide | 0.1 |
| Wax | 0.1 |

Manufacturing Process: Metformin and Silicon Dioxide were placed in a Glatt apparatus (GPCG1) and were sprayed with a PVA solution in water to prepare the granules. The granules are dried, passed through a sieve and blended with glyceryl behenate in a blender. The resulting mixture was compressed into tablets and coated with a coating solution comprising Ethyl Cellulose, Povidone and Dibutyl Sebacate, according to Table 9, in a coating pan (Air Flow 110 m3/hr, liquid flow 7 g/min at a temperature of 70′C at 2.9 bar pressure. The coated tablets were dried and coated with dapagliflozin as per the process described for Example 1.

The Example 6 was studied to determine the cumulative dissolution profiles using USP apparatus I (40 mesh baskets), 100 rpm, in 0.1N HCl, by taking 5-mL samples without media replacement, at 15 minutes, 30 minutes, and 1, 2, 4, 6, and 8 hours. The dissolution profile is shown in Table 12

TABLE 12

| Time Hours | Percent Dissolved |
|---|---|
| 0 | 0 |
| 2 | 51.1 |
| 4 | 70.4 |
| 6 | 84.2 |
| 8 | 94.1 |

Similarly, the dissolution profile the Example, as measured using a medium: 900 ml with phosphate buffer pH 6.8 at 75 rpm in a USP Apparatus I is shown in Table 13

TABLE 13

| Hours | Percent Dissolved |
|---|---|
| 0 | 0 |
| 2 | 13.1 |
| 4 | 33 |
| 8 | 65 |
| 12 | 86 |

### Example 8

In yet another example, a pharmaceutical dosage form comprising a slow release metformin and a sodium-dependent glucose transporter (SGLT2) inhibitor such as Sergliflozin was prepared according to composition in Table No 14:

TABLE 14

| | Amount mg/tablet |
|---|---|
| First Active Ingredient | |
| Metformin HCl | 500.0 |
| Povidone K 301 USP | 36.0 |
| Sodium Lauryl Sulfate | 25.8 |
| Magnesium Stearate | 2.8 |
| Seal Coat | |
| Opadry Clear (YS 1-7006) | 23.5 |
| Semi Permeable Coat | |
| Cellulose Acetate (398-10) NF | 21.5 |
| Triacetin | 1.4 |
| PEG 400 | 2.8 |
| Second Active Ingredient | |
| Sergliflozin | 5.0 |
| Tween | 1.4 |
| Polyplasdone XL | 11.2 |
| Opadry Clear (YS 1-7006) | 5.5 |

### Slow Release Biguanide and Slow Release (SGLT2) Inhibitor Combination;

Exemplary compositions of the pharmaceutical composition that comprises a slow release biguanide and a slow release sodium-dependent glucose transporter (SGLT2) inhibitor, and at least one pharmaceutically acceptable excipient can have the compositions described in Table 15:

TABLE 15

| First Slow Release Core | Percent of First Drug Core | Preferred Range % |
|---|---|---|
| Drug | 50-98% | 75-95% |
| Binder | 0.1-40% | 3-15% |
| Absorption Enhancer | 0-20% | 2-10% |
| Lubricant | 0-5% | 0.5-1% |
| Coat | Percent of Coat | |
| Polymer | 50-99% | 75-95% |
| Flux Enhancer | 0-40% | 2-20% |
| Plasticizer | 0-25% | 2-15% |
| Second Slow Release Core | Percent of Second Drug Core | |
| Drug | 50-98% | 75-95% |
| Binder | 0.1-40% | 3-15% |
| Absorption Enhancer | 0-20% | 2-10% |
| Lubricant | 0-5% | 0.5-1% |
| Coat | Percent of Coat | |
| Polymer | 50-99% | 75-95% |
| Flux Enhancer | 0-40% | 2-20% |
| Plasticizer | 0-25% | 2-15% |
| Final Slow Release Coat | Percent of Final Slow | |
| Optional | Release Coat | |
| Polymer | 50-99% | 75-95% |
| Flux Enhancer | 0-40% | 2-20% |
| Plasticizer | 0-25% | 2-15% |

The pharmaceutical composition prepared above exhibit the dissolution profile illustrated in Table 16 when tested as per United States Pharmacopeia XXIII, in a USP Type 2 apparatus at 75 rpm in 900 ml of simulated intestinal fluid (pH 7.5 phosphate buffer) and at 37° C.

TABLE 16

| | | |
|---|---|---|
| | | Preferred |
| Time Hours | Range percent | Range % |

Biguanide Release

| | | |
|---|---|---|
| 2 | 0-30% | 0-25% |
| 4 | 10-50% | 20-45% |
| 6 | 15-60% | 30-60% |
| 8 | 30-90% | 45-90% |
| 12 | More than 50% | More than 50% |
| 16 | More than 60% | More than 60% |
| 20 | More than 65% | More than 65% |

Sodium-dependent glucose transporter SGLT2 inhibitor Release

| | | |
|---|---|---|
| 2 | 0-30% | 0-25% |
| 4 | 10-50% | 20-45% |
| 6 | 15-60% | 30-60% |
| 8 | 30-90% | 40-90% |
| 12 | More than 50% | More than 50% |
| 16 | More than 60% | More than 60% |
| 20 | More than 65% | More than 65% |

### Example 9

### Preparation of Slow Release Metformin Hydrochloride and Slow Release Dapagliflozin Composition

A pharmaceutical composition comprising slow release dapagliflozin 10 mg and slow release metformin hydrochloride 850 mg, and at least one pharmaceutically acceptable excipient was manufactured as per the process outlined below in accordance with the formula of Table 17.

TABLE 17
Example 9; Slow Release Metformin 850 mg
plus Slow Release Dapagliflozin 10 mg

| | Amount mg |
|---|---|
| First Slow Release Core | |
| Metformin HCl | 850.0 |
| Povidone K 301 USP | 78.0 |
| Sodium Lauryl Sulfate | 51.7 |
| Magnesium Stearate | 5.7 |
| Seal Coat | |
| Opadry Clear (YS 1-7006) | 47.1 |
| Semi permeable coat | |
| Cellulose Acetate (398-10) NF | 15.8 |
| Triacetin | 0.9 |
| PEG 400 | 1.9 |
| Seal coat | |
| Opadry Clear (YS 1-7006) | 21.0 |
| Second Slow Release Core | |
| Dapagliflozin | 10 |
| Povidone K 301 USP | 4.0 |
| Sodium Lauryl Sulfate | 2.5 |
| Magnesium Stearate | 0.3 |
| Seal Coat | |
| Opadry Clear (YS 1-7006) | 2.4 |
| Semi permeable coat | |
| Cellulose Acetate (398-10) NF | 1.0 |
| Triacetin | 0.1 |
| PEG 400 | 0.1 |
| Seal coat | |
| Opadry Clear (YS 1-7006) | 1.1 |
| Final Slow Release Coat Optional | |
| Semi permeable coat | |
| Cellulose Acetate (398-10) NF | 19.0 |
| Triacetin | 1.1 |
| PEG 400 | 2.1 |
| Seal coat | |
| Opadry Clear (YS 1-7006) | 23.0 |

### Manufacturing Process:

A slow-release tablet comprising 850 mg of slow release metformin HCl and 10 mg slow release dapagliflozin was prepared using a three step process: 1) Preparation of First Slow Release Drug Core, 2) Preparation of Second Slow Release Drug Core and 3) Preparation of Slow Release Capsules.

Preparation of First Slow Release Drug Core: The slow release drug cores ware prepared by three step processes 1) Granulation, 2) Tableting and 3) Membrane coating. An optional seal coating may be applied to the core tablet. The preparation was carried out in accordance with the formula specified in Table 3. The specific steps are described below.

Granulation: Metformin hydrochloride was screened using a size reduction and screening equipment (Comil screener) and was further fluidized using a commercially available powder coater granulator (Glatt 60). It was sprayed with a povidone solution prepared in a steel tank using water as the solvent. The spraying was carried at about 2.5 bar pressure by varying the pump rate from 0-15 minutes for a target of 500 g/min. to achieve a target of about 1200 g/min. in the final phase. Granules were dried until an LOD of less than 2% and passed through a screener (Comil 1143/75).

Tableting: The coated metformin hydrochloride was mixed with sodium lauryl sulfate in a blender (Slant-Cone: 30 minutes). Magnesium stearate was screened and blended with the metformin hydrochloride-sodium lauryl sulfate mixture. The homogenized mixture was compressed into cores using standard procedures. The metformin hydrochloride tablets weighted from 800 mg to 1300 mg with a frigidity of less than 1%.

Seal coating: Seal coating of the metformin core cores was accomplished by spraying (O'Hara Lab Coat Pan Coater) a solution of either Opadry coating material. The spraying was conducted at a temperature of 46-47° C., atomization pressure of 40-60 psi at a spray rate of 180 grams per minute/three guns. The pan speed was at 4-8 rpm and air volume of 1000+/−100. The seal coated metformin hydrochloride tablets had a theoretical coating of 2.5-5.0 weight %.

Membrane coating: Cellulose acetate was mixed with acetone to prepare a clear solution. Polyethylene glycol 400 was added this mixture and triacetin was added to the resulting solution. The seal coated metformin hydrochloride cores were fluidized using a Glatt coater. The cellulose acetate solution was sprayed onto the fluidized seal coated metformin hydrochloride cores at an atomization pressure of 2.5 bars, using an air volume of 1700 CFM, at spraying rate of about 450 g/ml to achieve coating target of 1.3 weight %. The membrane coated tablets were dried sequentially at temperature of 21° C. and 40° C. An orifice was made on the membrane coated tablets using laser with an average diameter of 0.4 to 0.5 mm with micrometer ranging from 6 to 7 mm. Laser was operated with pulse width of 165+/−65 and a pulse delay of around 340+/−100 respectively.

Preparation of Second Slow Release Drug Core: The second slow release drug core comprising dapagliflozin was prepared according to the process described above for the first slow release drug core by replacing metformin hydrochloride with dapagliflozin and modifying the other constituents according Table 14.

Membrane coating: Cellulose acetate was mixed with acetone to prepare a clear solution. Polyethylene glycol 400 was added this mixture and triacetin was added to the resulting solution. The seal coated dapagliflozin cores were fluidized using a Glatt coater. The cellulose acetate solution was sprayed onto the fluidized seal dapagliflozin cores at an atomization pressure of 2.5 bars, using an air volume of 1700 CFM, at a spraying rate of about 450 g/ml to achieve coating target of 1.3 weight %. The membrane coated tablets were dried sequentially at temperature of 21° C. and 40° C. An orifice was made on the membrane coated tablets using laser with an average diameter of 0.4 to 0.5 mm with micrometer ranging from 6 to 7 mm. Laser was operated with pulse width of 165+/−65 and a pulse delay of around 340+/−100 respectively.

### Preparation of Final Slow Release Metformin and Slow Release Dapagliflozin Drug

The First Slow Release Drug Core and the Second Drug Release Core were placed in a hard gelatin capsule commercially available from Capsugel and can be optionally further coated with a final slow release coating.

Optionally, color coated capsules were dried and polished using Cindrella wax and the finished final drugs were packaged in a HDPE bottle with a suitable desiccant and subjected appropriate stability and clinical studies.

The pharmaceutical composition prepared as per Example 8 exhibit the dissolution profile illustrate in Table 18 when tested as per United States Pharmacopeia XXIII, in a USP Type 2 apparatus at 75 rpm in 900 ml of simulated intestinal fluid (pH 7.5 phosphate buffer) and at 37° C.

TABLE 18
Dissolution Profile 850/10 mg Tablets

| Time hours | Metformin HCl | Dapagliflozin |
|---|---|---|
| 0 | 0.00% | 0.00% |
| 2 | 18.00% | 21.00% |
| 4 | 31.00% | 38.00% |
| 8 | 58.00% | 59.00% |
| 12 | 78.00% | 78.00% |
| 16 | 90.00% | 90.00% |
| 20 | 92.00% | 91.00% |

### Example 10

### Preparation of Slow Release Metformin Hydrochloride Plus Slow Release Dapagliflozin Plus an Immediate Release Dapagliflozin Composition

Exemplary compositions of the pharmaceutical composition that comprises a slow release core comprising a biguanide and a sodium-dependent glucose transporter (SGLT2) inhibitor, and at least one pharmaceutically acceptable excipient and an immediate release layer comprising a sodium-dependent glucose transporter (SGLT2) inhibitor, using dapagliflozin are described before for illustrative purposes:

The slow release metformin hydrochloride 500 mg plus slow release dapagliflozin 5 mg plus an immediate release dapagliflozin 5 mg composition was prepared according the formula in Table 16 in two step processes; 1) Preparation of Slow Release Metformin Hydrochloride and Slow Release Dapagliflozin and 2) Coating of Immediate Release Dapagliflozin layer.

### Preparation of Slow Release Metformin Hydrochloride and Slow Release Dapagliflozin;

The combination comprising a slow release metformin and Slow Release dapagliflozin was prepared as a bilayer tablet as exemplified below in Table 19:

TABLE 19
Example 10; Bilayer Tablets (Slow Release Metformin
500+ Slow Release Dapagliflozin 5 mg)
Layer 1

| Metformin Hydrochloride | 500 mg |
|---|---|
| Microcrystalline cellulose | 10-25% |
| Polyvinyl alcohol | 3-5% |
| Ethylcellulose (5-20 cp) | 10-20% |
| Hydroxyethyl cellulose | 5-15% |
| Colloidal silicon dioxide | 2-5% |
| Sodium stearyl fumarate | 1-2% |

Layer 2:

| | |
|---|---|
| Dapagliflozin | 5 mg |
| Microcrystalline cellulose | 5-20% |
| Povidone | 10-15% |
| Crosscarmellose sodium | 5-10% |
| Magnesium stearate | 0.5-2% |

Preparation of Layer 1: Metformin Hydrochloride, microcrystalline cellulose and colloidal silicon dioxide were granulated with polyvinyl alcohol and dried. The dried granules are mixed with Ethylcellulose and Hydroxyethylcellulose and lubricated with Sodium stearyl fumarate.

Preparation of Layer 2: Dapagliflozin mixed with microcrystalline cellulose was granulated with povidone. Granules are dried and mixed with Crosscarmellose sodium and finally lubricated with Magnesium stearate.

Compression: Layer 1 and Layer 2 are loaded into the hopper of Bilayer rotary compression machine and compressed with a desired hardness.

The final composition comprising a Slow Release Metformin 500 plus Slow Release Dapagliflozin 5 mg plus Immediate Release Dapagliflozin 5 mg will have the following formula of Table 20:

TABLE 20
Example 10; Slow Release Metformin 500
plus Slow Release Dapagliflozin 5 mg plus
Immediate Release Dapagliflozin 5 mg

Weight or Percent or Layer

Layer 1

| | |
|---|---|
| Metformin Hydrochloride | 500 mg |
| Microcrystalline cellulose | 10-25% |
| Polyvinyl alcohol | 3-5% |
| Ethylcellulose (5-20 cp) | 10-20% |
| Hydroxyethyl cellulose | 5-15% |
| Colloidal silicon dioxide | 2-5% |
| Sodium stearyl fumarate | 1-2% |

Layer 2:

| | |
|---|---|
| Dapagliflozin | 2.5 mg |
| Microcrystalline cellulose | 5-20% |
| Povidone | 10-15% |
| Crosscarmellose sodium | 5-10% |
| Magnesium stearate | 0.5-2% |

Immediate Release Layer

| | |
|---|---|
| Dapagliflozin | 2.5 mg |
| Opadry White | 23 |
| Tween | 2 |
| Acetone/Water | Q.S |

The illustration is representative and Dapagliflozin can be substituted by another SGLT2 inhibitor such as Remogliflozin and Sergliflozin etc.

Manufacturing process of dapagliflozin coating;

The slow release metformin hydrochloride 500 mg plus slow release dapagliflozin 5 mg. prepared above was coated with an immediate release layer comprising dapagliflozin 5 mg as below

Prepare the dapagliflozin dispersion by taking required quantity of purified water in a SS vessel with stirrer. Dissolve tween-80 in it and disperse Opadry white under stirring till a uniform dispersion is prepared. Add dapagliflozin into Opadry white suspension and mixed thoroughly till a uniform dispersion is formed. Circulate this dispersion through a colloid mill with a minimum clearance of 0.2 mm to form a homogenous dispersion. Load the Cellulose acetate coated tablets in coating pan and coat the tablets with dapagliflozin dispersion with inlet temp of 65-70 deg. C., exhaust temp of 45-48 deg. C., atomization pressure of 2 Bar. 2% excess is considered for dapagliflozin per tablet in order to consider the losses during coating. Apply a seal coat on dapagliflozin coated tablets of about 1% using Opadry clear (HPMC coat). Dry the tablets at 35-40 deg. C. for 15 minutes and allowed to cool the tablets in coating pan by rotating the pan intermittently. Unload the tablets and packed the packed in double polythene lined well closed containers.

The Example 10 had the following dissolution profile for the metformin hydrochloride when tested as per United States Pharmacopeia XXIII, in a USP Type 2 apparatus at 75 rpm in 900 ml of simulated intestinal fluid (pH 7.5 phosphate buffer) and at 37° C.

Dissolution Profile of slow release metformin 500 mg plus slow release dapagliflozin phosphate 5 mg plus Immediate Release dapagliflozin 5 mg Tablets as per Table 21.

TABLE 21

| Time hours | Metformin HCl |
|---|---|
| 0 | 0.00% |
| 2 | 18.00% |
| 4 | 31.00% |
| 8 | 58.00% |
| 12 | 78.00% |
| 16 | 90.00% |
| 20 | 92.00% |

The invention is now described with reference to the above Examples. Without further description, it is believed that one of ordinary skill in the art can, using the preceding description and the following illustrative examples, make and utilize the disclosed compositions. The above working examples therefore, are provided for the purpose of illustration only and specifically point out the preferred embodiments, and are not to be construed as limiting in any way the remainder of the disclosure. Therefore, the examples should be construed to encompass any and all variations which become evident as a result of the teaching provided herein.

### Reference Example 1

### Preparation of Immediate Release Dapagliflozin Tablets;

As a reference example, an Immediate Release Dapagliflozin tablets were prepared using standard manufacturing process as the formula in Table 22.

TABLE 22
Reference Example

| Ingredients | mg/tablet |
|---|---|
| Dapagliflozin | 5 |
| Pregelatinized Starch | 7.5 |
| Microcrystalline Cellulose | 34.3 |
| Sodium Glycolate Starch | 1.5 |
| Silicon Dioxide | 1 |
| Magnesium Stearate | 1 |

### Example 11

### Preparation of Slow Release Dapagliflozin and Immediate Release Metformin Hydrochloride

The pharmaceutical composition comprising a slow release Sitagliptin 50 mg and immediate release metformin hydrochloride 500 mg and at least one pharmaceutically acceptable excipient was manufactured according to standard procedures in accordance with the formula of Table 23.

TABLE 23
Example 11; Slow Release Dapagliflozin 5
mg plus Immediate Release Metformin 500

| | mg/tablet |
|---|---|
| Core | |
| Dapagliflozin | 5 |
| Microcrystalline Cellulose | 57 |
| Sodium Stearyl Furmarate | 1 |
| Slow Release Coat | |
| Talc | 6 |
| Sodium Stearyl Furmarate | 2 |
| Opadry | 10 |
| Eudragit S 500 | 15 |
| Immediate Release Layer | |
| Metformin Hydrochloride | 500 |
| Lactose | 375 |
| Povidone | 5.2 |
| Lutrol | 5 |
| Water | Q.S |
| Total Weight | 1026.2 |

The Example 4 was tested in vivo each in a cross over study with the combination of Glucophage XR 500 mg×2 tablets (commercially available metformin XR tablets) and Dapagliflozin 5 mg×2 (Reference Example 1). The in vivo test employed 14 healthy volunteers and each dosed after evening meal.

The Pharmacokinetic parameters of metformin hydrochloride and dapagliflozin are listed in Table 24 and Table 25 respectively

TABLE 24
Metformin Parameter

| | | | | AUC | |
|---|---|---|---|---|---|
| | | Mean | Mean | Ratio | Cₘₐₓ |
| | Mean AUC0-12 | Cₘₐₓ | Tₘₐₓ | (Test/ | (Test/ |
| Drug/day | (ng · hr/ML) | (ng/ML) | hr | BID) | BID) |
| Glucophage 500 mg X 2 | 9899 | 1361 | 3.5 | 1 | 1 |
| Example 4 | 10256 | 1378 | 7.3 | 1.03 | 1.01 |

TABLE 25
Dapagliflozin Parameter

| | Mean | Mean | Mean | AUC Ratio | Cₘₐₓ |
|---|---|---|---|---|---|
| | AUC0-12 | Cₘₐₓ | Tₘₐₓ | (Test/ | (Test/ |
| Combination drug/day | (ng · hr/ML) | (μg/ML) | hr | BID) | BID) |
| Dapagliflozin (Ref Ex 1) 5 mg X2 | 8.43 | 0.73 | 1.6 | 1 | 1 |
| Example 4 | 8.3 | 0.75 | 1.7 | 0.98 | 1 |

The results demonstrate that metformin and dapagliflozin didn't have any impact on each other pharmacokinetic parameters when administered as a fixed dose combination of instant invention. The results also demonstrate that a fixed dose combination of a slow release biguanide and an SGLT2 inhibitor is bioequivalent to a co-administered dosage form comprising equivalent doses of a slow release biguanide and an SGLT2 inhibitor.

### Example 12

### Method of Administration

The compositions disclosed were administered to patients using a controlled human clinical trial. The study determined the efficacy of sodium-dependent glucose transporter (SGLT2) inhibitor, biguanide alone and a combination of a sodium-dependent glucose transporter (SGLT2) inhibitor and a slow release biguanide; for example metformin for the treatment of non-insulin dependent diabetes mellitus (NIDDM). The trial was designed to target a segment of the type 2 diabetes population wherein the disease state has progressed to a point where maximum doses of metformin are usually required. The patients chosen were at a stage where the stimulated pancreatic insulin secretion does not keep up with the increasing demand. Since the un-stimulated (metformin) insulin secretory capacity of the beta cells is very low in this population, a reversal of insulin resistance alone would be of partial benefit. Therefore, maintaining a level of stimulated insulin secretion with a metformin while adding a sodium-dependent glucose transporter (SGLT2) inhibitor to improve insulin sensitivity could provide a level of glycemic control unattainable by either medication alone.

A primary objective of the study was to assess the efficacy of a sodium-dependent glucose transporter (SGLT2) inhibitor in combination with a slow release biguanide in patients with type 2 diabetes by comparing changes in markers of glycemic and lipid homeostasis over six months of treatment.

The effect of treatment on the pattern of post-prandial glucose tolerance (standard 2-hour meal tolerance test) was determined in a subset of patients.

### Clinical Trial I: Slow Release Metformin and Dapagliflozin Protocol

TITLE: A 24 weeks, Prospective, Open, Randomized, Comparative, Three-Arm, Parallel-Group Study To Evaluate The Efficacy And Tolerability Of 1) Fixed dose combination (FDC) of Dapagliflozin 5 mg and Metformin slow release 500 mg (Example 1)-Two Tablets and 2) Glucophage XL 500 mg (2 tablets), 3) Dapagliflozin 5 mg (2 tablets), all drugs administered once daily orally for their blood glucose lowering effect in patients with type-2 diabetes mellitus who are inadequately controlled on Metformin 1500 mg daily for 4 weeks.
SAMPLE SIZE: A total of 66 patients were enrolled, assigned about 20 in each of the three treatment arms.
INVESTIGATION DRUGS: 1) Fixed dose combination (FDC) of Dapagliflozin 5 mg and Metformin slow release 500 mg (Example 4)-Two Tablets and 2) Glucophage XL 500 mg (2 tablets), 3) Dapagliflozin 5 mg (2 tablets), all drugs administered once daily orally.
INDICATION(s): Patients with type-2 diabetes mellitus who are inadequately controlled on Metformin 1500 mg daily.
STUDY DESIGN: This was a 24 weeks, open, randomized, controlled, multi-center, parallel run, efficacy & tolerability study designed to evaluate the efficacy of 1) Fixed dose combination (FDC) of Dapagliflozin 5 mg and Metformin slow release 500 mg (Example 4)-Two Tablets and 2) Glucophage XL 500 mg (2 tablets), 3) Dapagliflozin 5 mg (2 tablets), all drugs administered once daily orally, for their blood glucose lowering effect in patients with type-2 diabetes mellitus who are inadequately controlled on Metformin 1500 mg daily for 4 weeks.
PRIMARY OBJECTIVE: Was to compare the efficacy of 1) Fixed dose combination (FDC) of Dapagliflozin 5 mg and Metformin slow release 500 mg (Example 4)-Two Tablets and 2) Glucophage XL 500 mg (2 tablets), 3) Dapagliflozin 5 mg (2 tablets), all drugs administered once daily orally, for their blood glucose lowering effect in patients with type-2 diabetes mellitus who are inadequately controlled on Metformin 1500 mg daily for 4 weeks. This was carried out by:
   Monitoring the glycosylated hemoglobin (HbA1c) and fasting plasma glucose.
   Samples for HbA1c & glucose will be taken at Screening (V1), Baseline (V2), 2 Weeks (V3), 4 Weeks (V4), 5 Weeks (V5) and 6 Weeks (V6).
SECOND OBJECTIVE: Was to compare the tolerability of 1) Fixed dose combination (FDC) of Dapagliflozin 5 mg and Metformin slow release 500 mg (Example 4)-Two Tablets and 2) Glucophage XL 500 mg (2 tablets), 3) Dapagliflozin 5 mg (2 tablets), all drugs administered once daily orally, for their blood glucose lowering effect in patients with type-2 diabetes mellitus who are inadequately controlled on Metformin 1500 mg daily for 4 weeks. This was carried out by:
   Documenting the number and seriousness of hypoglycemic events,
   Documenting the drop-out rate,
   Documenting hematological, liver and renal function and lipid, parameters at Screening, Baseline and study conclusion (4 weeks), and
   Monitoring AEs throughout the study.

Patients overall satisfaction was assessed by standard Diabetes Treatment Satisfaction Questionnaire (DTSQc)

### Diagnosis and Key Subjects Selection Criteria:

Subjects were male or female between the age group of 18 to 75 years, both inclusive, with at least a 1-year history of Type-2 Diabetes Mellitus not controlled by oral Metformin 1500 mg daily for at-least 12 weeks. Subjects must otherwise be in good general health.

### Inclusion Criteria:

Subjects satisfied all of the following inclusion criteria to participate in the study—
1. Was a male or female between the ages 18 to 75 years;
2. Had at least a 1-year history of Type-2 DM;
3. Was inadequately controlled diabetes with Metformin 1500 mg per day for at-least 12 weeks prior to screening and not receiving any other oral anti-diabetic agent(s);
4. On current physical examination, vital signs or ECG at screening that reveals no clinically significant abnormalities;
5. Had a body mass index (BMI) between 25 to 45 kg/m2 both inclusive;
6. Had a glycosylated hemoglobin HbA1c between 7 to 10% both inclusive;
7. Was willing to follow the American Diabetes Association or the International Diabetes Federation diet guidelines for Type-2 Diabetes Mellitus; was able to record hypoglycemic symptoms and other adverse events;
8. Provided written informed consent prior to admission into the study; and
9. If female of childbearing potential, used a reliable form of birth control and are willing to continue as such for the duration of the study.

### Exclusion Criteria:

Patients excluded from the study if they meet any of the following exclusion criteria:
1. Had a history suggestive of, or presence of significant cardiac, gastrointestinal, endocrine, neurological, liver, or kidney disease, or conditions known to interfere with the absorption, distribution, metabolism, or excretion of study drugs;
2. Had a history of drug or alcohol dependency or psychological disease;
3. Required regular use of medication (other than study medication) that interferes with the absorption and/or metabolism of study drugs; subjects on concomitant medications that alter blood glucose levels (e.g., steroids);
4. Participated in a clinical trial or use of an investigational drug within 30 days prior to admission to this study;
5. Had an episode of severe hypoglycemia with seizure or coma within the past year;
6. Had a diagnosis of Type-1 Diabetes Mellitus;
7. Were on Insulin therapy within one year;
8. Had a history of ketoacidosis within 6 months prior to admission to this study;
9. Had a history of myocardial infarction, coronary artery bypass surgery, post-transplantation cardiomyopathy or stroke within the previous 6 months;
10. Had any acute illness within 2 weeks prior to Screening;
11. Had elevated liver enzymes (ALT, AST, alkaline phosphatase), as follows: if values for any two of the liver enzymes is >3 times the upper limit of normal;
12. Had elevated renal parameters (Blood urea nitrogen & serum creatinine), as follows: if value for any of the parameters is >3 times the upper limit of normal;
13. Subjects who had participated in any clinical trial or use of an investigational drug within 30 days prior to admission to this study; or
14. Was a pregnant or lactating female patient.

STUDY DESIGN & PROCEDURES: This was a 24 week, open, randomized, controlled, multi-center, parallel run, efficacy & tolerability study designed to evaluate the efficacy of 1) Fixed dose combination (FDC) of Dapagliflozin 5 mg and Metformin slow release 500 mg (Example 4)-Two Tablets and 2) Glucophage XL 500 mg (2 tablets), 3) Dapagliflozin 5 mg (2 tablets), all drugs administered once daily orally, for their blood glucose lowering effect in patients with type-2 diabetes mellitus who are inadequately controlled on Metformin 1500 mg daily for 12 weeks

Patients were required to make 4 visits during the study period. After a Screening visit (V1) to determine eligibility, each subject will return at the baseline visit (V2), where they will be instructed about the dosing schedules & diet.

Subjects were required take the study medications as tablets administered orally two times in a day in the morning before breakfast and at bedtime with a glass of water.

All subjects were monitored by the Investigators and/or by the study coordinator by phone and regular clinic visits.

At every scheduled visit, subjects reported their general well being and any reported AEs. If subjects had problems or if there was a continuous deterioration of fasting plasma glucose or patient condition without known clinical reasons, the investigator reassessed the subject to determine if they could continue with the study.

If the subjects are terminated from the study, the subject was followed by the investigator to assure proper medical care was provided, and once stable, returned to the primary health care provider.

The following procedures were carried out during the study:

Screening (Visit 1, Day -10 to -2):
Study related procedures were explained and informed consent was taken.
Detailed medical history was collected.
Demographic data was collected.
An abbreviated physical examination including weight and vital signs (blood pressure, heart rate, temperature, respiration rate) was conducted.
Vital signs were obtained after the patient has been in a supine position for at least 5 minutes.
Fasting blood samples for plasma glucose, serum insulin, C-peptide & lipid profile was collected.
Blood samples were collected for hematological, liver function test, renal function, & urine analysis will be done.
Thyroid function (TSH), HIV status, 12 lead ECG was performed.
Serum pregnancy tests in women of child-bearing potential were performed.
Administrated the checklist for Inclusion/exclusion criteria.

### Baseline (Visit 2, Day -2 to 1):

Physical examination was conducted.
Vital signs were evaluated.
Fasting blood samples for plasma glucose, serum insulin, C-peptide & lipid profile were collected.
Blood samples collected and hematological, liver function test, renal function, & urine analyses were done.
Baseline AEs (if any) were recorded.
Administrated the checklist for inclusion/exclusion criteria.
Drugs were dispensed and diet instructions will be given.
Patient diary was given to patient and instructions were given for filling the diary.

### Week 6 (Visit 3):

Physical examination was conducted.
Vital signs were recorded.
Fasting blood samples for plasma glucose, serum insulin, C-peptide, & lipid profile were collected.
Blood samples collected and fasting plasma glucose & urine analyses were done.
All AEs & SAE's (if any) were recorded and necessary action was taken.
Patient compliance for diet & medication were recorded by interview and tablet count.
Drugs were dispensed and diet instructions were given.

### Week 12 (Visit 4):

Physical examination was conducted.
Vital signs were recorded.
Fasting blood samples for plasma glucose, serum insulin, C-peptide, & lipid profile were collected.
Blood samples collected and fasting plasma glucose & urine analyses were done.
All AEs & SAE's (if any) were recorded and necessary action was taken.
Patient compliance for diet & medication were recorded by interview and tablet count.
Drugs were dispensed and diet instructions were given.

### Week 18 (Visit 5):

Physical examination was conducted.
Vital signs were recorded.
Fasting blood samples for plasma glucose, serum insulin, C-peptide, & lipid profile were collected.
Blood samples collected and fasting plasma glucose & urine analyses were done.
All AEs & SAE's (if any) were recorded and necessary action was taken.
Patient compliance for diet & medication were recorded by interview and tablet count.
Drugs were dispensed and diet instructions were given.

### Week 24 (Visit 6):

Physical examination was conducted.
Vital signs were recorded.
Fasting blood samples for plasma glucose, serum insulin, C-peptide & lipid profile were collected.
All AEs & SAE's (if any) recorded and necessary action was taken if needed.
Patient compliance for diet & medication was recorded by interview and tablet count.
Diabetes Treatment Satisfaction Questionnaire (DTSQ) will be filled by patient.

Over the course of the study, subjects consumed regular meals as suggested by the National Cholesterol Education Program (NCEP) ATP III (Adult Treatment Panel III) in therapeutic life style changes (TLC) nutrition component. All adverse events were recorded in the patient diary throughout the study and evaluated by the investigator upon Site Visits.

### Outcome Measures:

### Primary Outcome Measure:

Percent change in HbA1C from baseline after 24 weeks of treatment.
   Secondary Outcome Measures (after 24 weeks):
Percent change in fasting plasma glucose,
Percent change in body weight,
Responder rates for HbA1C (target<7%);
Responder rates for body weight (target BMI<25 kg/m2), and
Change from baseline in lipid profile.

### Safety Measures:

Physical examination.
Vital Signs.
Reporting of Adverse Events (AE's) & Serious Adverse Events (SAE's).
Abnormal laboratory values of laboratory safety parameters.

### Treatments:

### Investigational Treatment:

FDC containing Dapagliflozin 5 mg plus slow release Metformin 500 mg (Example 1) two tablets administered once daily.

### Comparative Treatment:

1. Metformin XL 500 mg two tablets administered once daily.

2. Dapagliflozin 5 mg two tablets administered once daily.

### Statistics

Sample Size: As this was a pilot study, sample size is not based on any statistical calculations.

Analysis Populations: Analysis populations included the per-protocol (PP) population & intention to treat (ITT) population.

Data expression: All parametric data expressed as Mean±1 S.D. (1 Standard Deviation). Proportions are expressed as numbers & percentages. For all statistical tests, the significance level were taken as p<0.05 at 95% C.I.

Data Analysis: No interim analysis was planned for this study.

Normality testing was be done using Kolmogorov-Smirnov test, if data found to be normal, One-Way ANOVA was used for comparison of multiple treatments for change in HbA1C, fasting plasma glucose, body weight & lipid profile. Post-hoc multiple comparisons would be made for investigational treatment vis-a-vis the 5 comparator treatments using Tukey's test.

Responder rates & proportions were tested using Chi-square test.

The patient characteristics are listed below in Table 26;

TABLE 26
Patient Characteristics

| | Dapagliflozin | Glucophage | FDC (Dapagliflozin 5 mg + |
|---|---|---|---|
| Characteristic/Treatment | 5 mg X 2 | XR 500 mg X 2 | Metformin XL 500 mg) X 2 |
| N | 22 | 21 | 22 |
| Mean age +/− SD (y) | 43 ± 3.2 | 45 ± 4.8 | 47 ± 3.4 |
| Sex (M:F) | 11; 11 | 10; 11 | 10; 12 |
| BMI | 33 ± 3.1 | 32 ± 3.3 | 33.5 ± 3.8 |
| Mean HbAlc (%) | 9.6 | 9.7 | 9.7 |
| Mean FPG (mg/dl) | 237.5 | 235.6 | 243.2 |
| Disease Duration (Years) | 6.5 | 6.3 | 7.0 |

### Biostudies

A total of 30 subjects were enrolled with fifteen subjects in each of the two studies as listed below and all of them randomly received from Example 9, Example 10 and Reference Example 1.

Study 1: Slow Release Metformin+Slow Release Dapagliflozin+Immediate Release Dapagliflozin—Each patient randomly received one dose of Reference Example 1 (Dapagliflozin 5 mg, Example 10 (Slow Release Metformin 500 mg+Slow Release Dapagliflozin 2.5 mg+Immediate Release Dapagliflozin 2.5 mg).

Study 2: Slow Release Metformin+Slow Release Dapagliflozin—Each patient randomly received one dose of Reference Example 1×2 tablets or Example 9 (Slow Release Metformin 850 mg+Slow Release Dapagliflozin 10 mg×1 Tablet).

The each study included four treatment phases wherein each phase was separated by washout period of 21 days. Subjects were randomized to receive one of the above drugs as randomly assigned by Latin Square and each subject crossed to over to next regimen according to the randomization sequence until all subjects have received all regimens (with twenty one week separating each regimen). Blood samples were centrifuged within 2 hours of collection and the plasma were separated and frozen at −10′ C or lower until assayed and pharmacokinetics data were calculated.

### Results

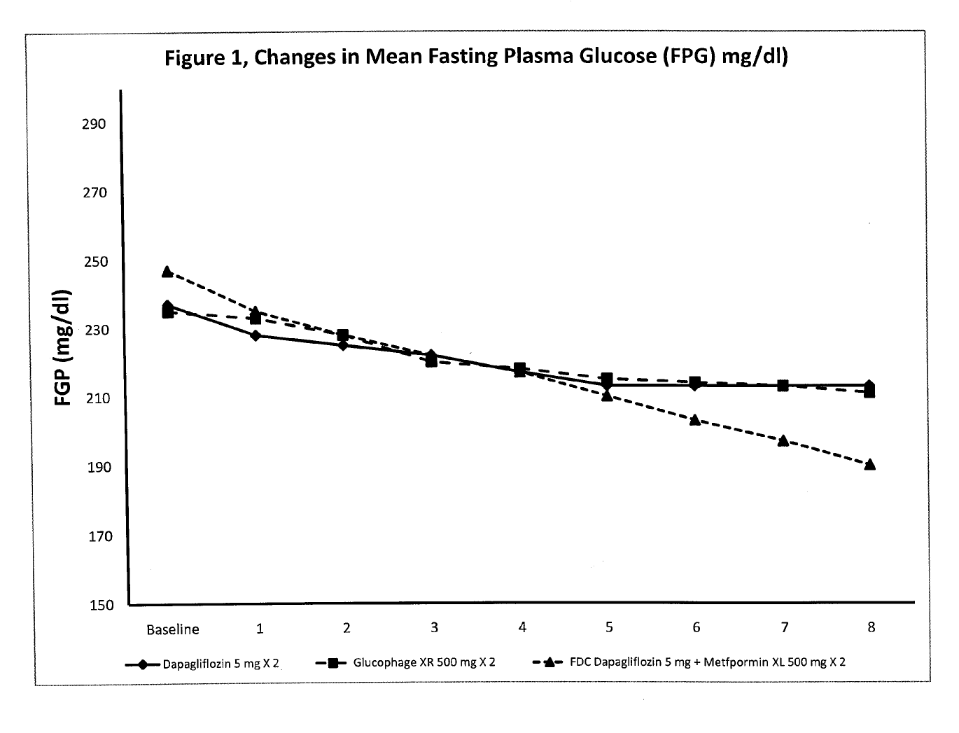

FIG. 1: This figure illustrates the changes in fasting plasma glucose (FPG) (+/−−) SEM) during administration of: 1) a fixed dose combination (FDC) of Dapagliflozin 5 mg and slow release Metformin 500 mg (Example 1, Two Tablets), 2) Glucophage XR 500 mg (2 tablets) and 3) Dapagliflozin 5 mg (Reference Example I, Two Tablets), all drugs administered once daily orally.

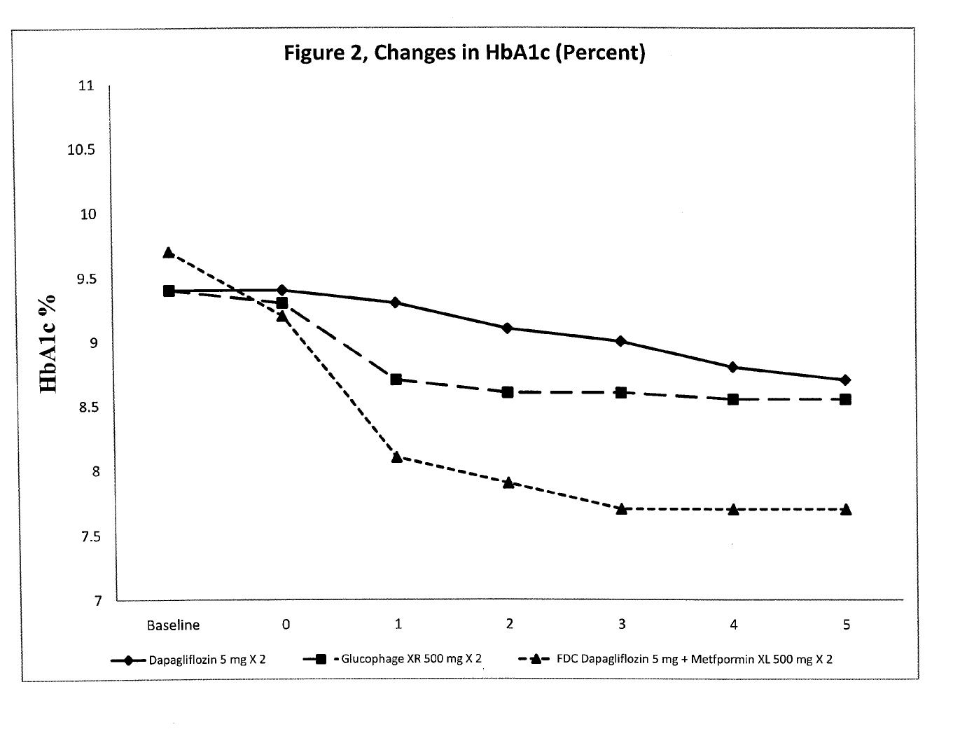

FIG. 2: This figure illustrates the changes in hemoglobin A1c (HbA1c) (+/−.SEM) during administration of: 1) a fixed dose combination (FDC) of Dapagliflozin 5 mg and slow release Metformin 500 mg (Example 1, Two Tablets), 2) Glucophage XR 500 mg (2 tablets) and 3) Dapagliflozin5 mg (Reference Example 1, Two Tablets), all drugs administered once daily orally.

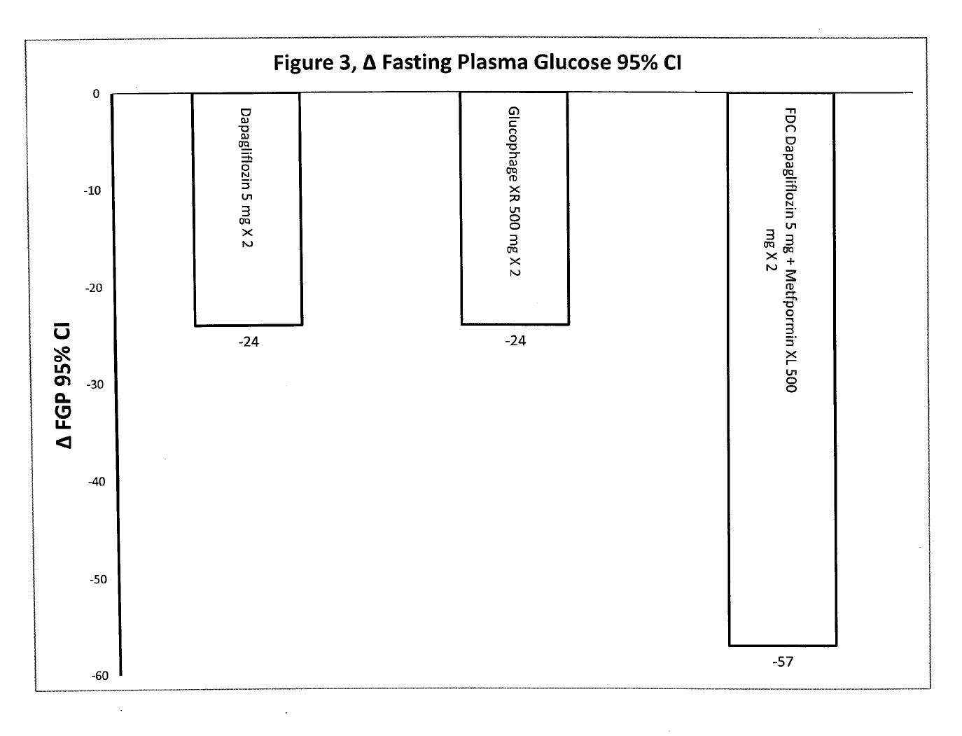

FIG. 3: This figure illustrates the Mean (A) Changes in fasting plasma glucose (FPG) (+/−−) SEM) during administration of: 1) a fixed dose combination (FDC) of Dapagliflozin 5 mg and slow release Metformin 500 mg (Example 1, Two Tablets), 2) Glucophage XR 500 mg (2 tablets) and 3) Dapagliflozin 5 mg (Reference Example 1, Two Tablets), all drugs administered once daily orally.

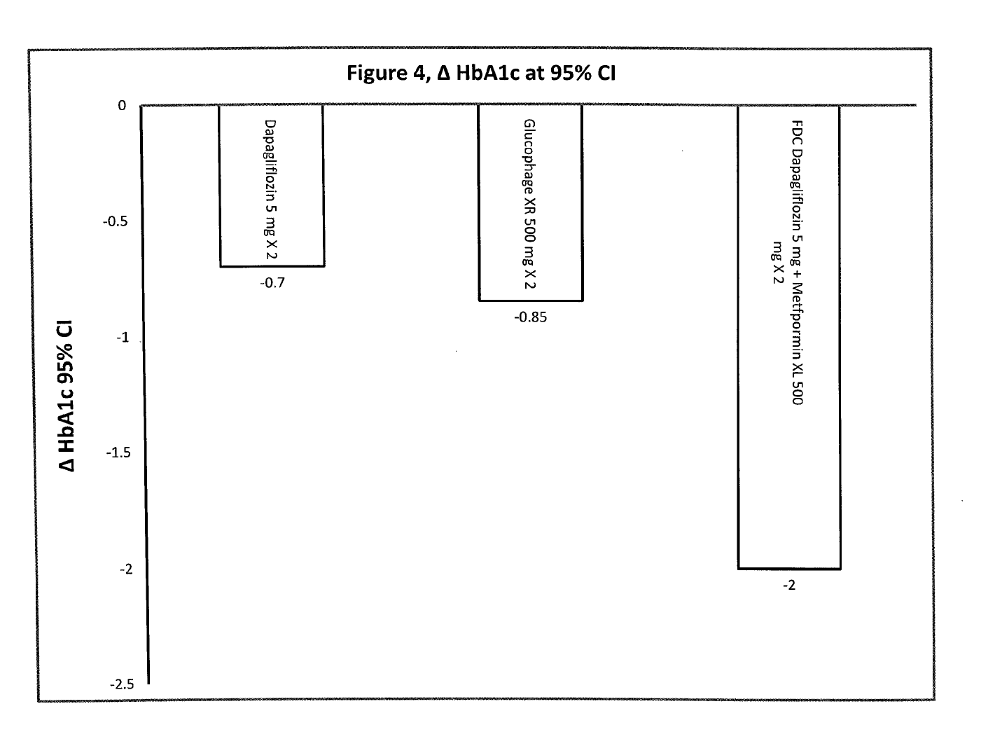

FIG. 4: This figure illustrates the Mean (A) Changes in hemoglobin A1c (HbA1c) (+/−.SEM) during administration of: 1) a fixed dose combination (FDC) of Dapagliflozin 5 mg and slow release Metformin 500 mg (Example 1, Two Tablets), 2) Glucophage XR 500 mg (2 tablets) and 3) Dapagliflozin 5 mg (Reference Example 1, Two Tablets), all drugs administered once daily orally.

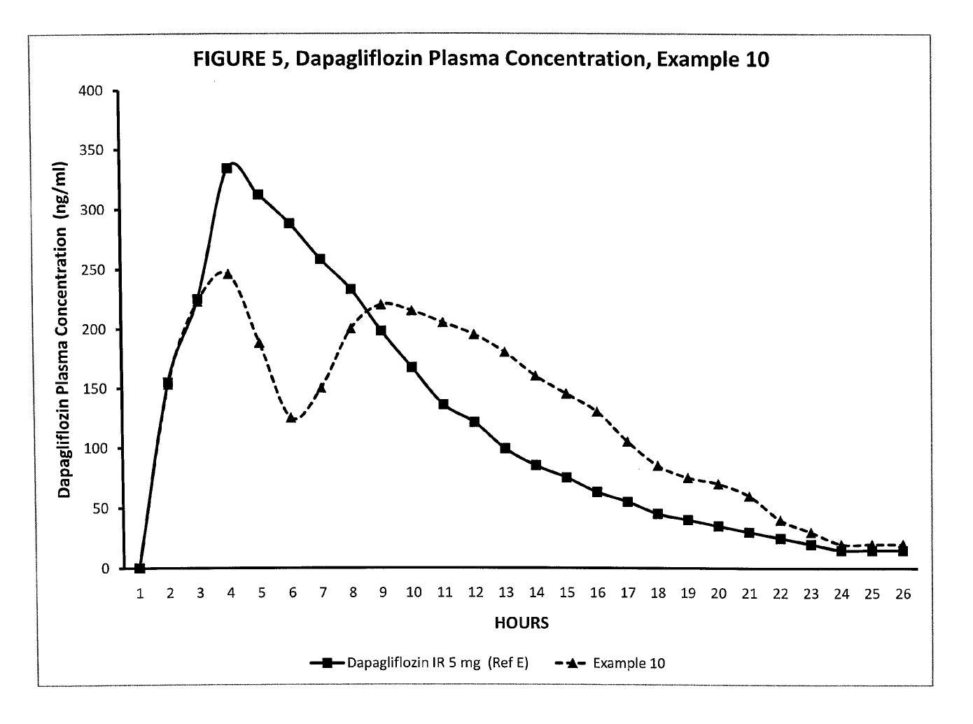

FIG. 5 is a graph that illustrates the mean plasma concentration of dapagliflozin after administration of single dose of Reference Example 1 dapagliflozin 5 mg and Example 10 (Slow Release Metformin 500 plus Slow Release Dapagliflozin 2.5 mg plus Immediate Release Dapagliflozin 2.5 mg).

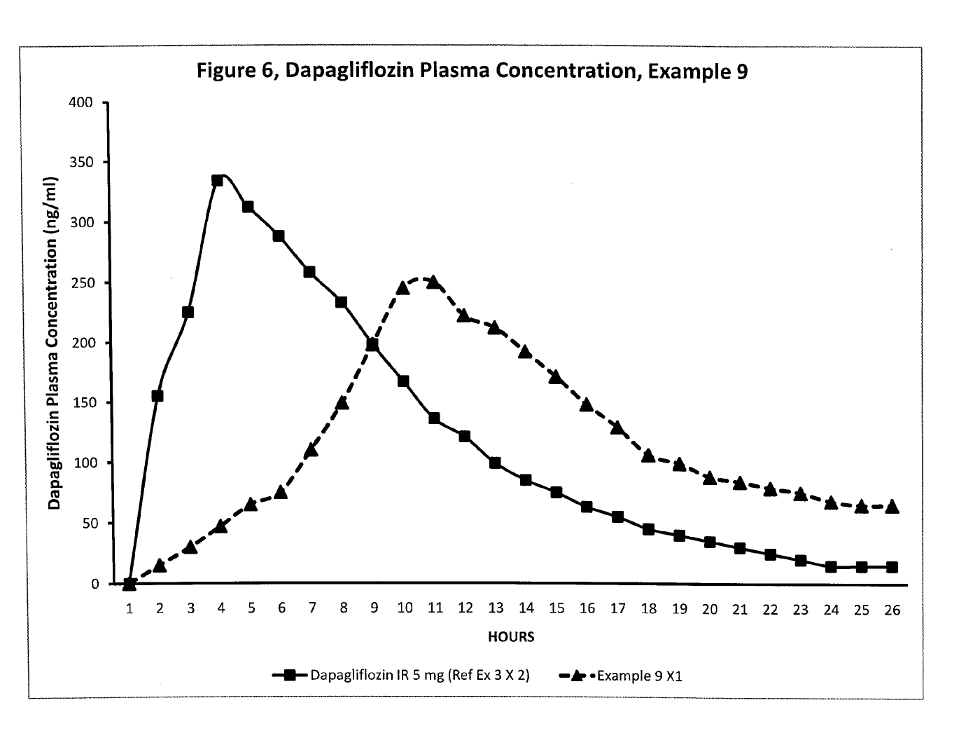

FIG. 6 is a graph that illustrates the mean plasma concentration of dapagliflozin after administration of single dose of Reference Example 1—Dapagliflozin 5 mg×2 and Example 9 (Slow Release Metformin 850 plus Slow Release Dapagliflozin 10 mg).

The abbreviations used herein have their conventional meaning within the chemical and biological arts. The disclosures of each and every patent, patent application, and publication cited herein are expressly incorporated herein by reference in their entirety into this disclosure. All publications, patents, and patent documents cited in the specification are incorporated by reference herein, as though individually incorporated by reference. In the case of any inconsistencies, the present disclosure, including any definitions therein will prevail. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

1. A pharmaceutical composition comprising at least two active agents;
wherein the active agents comprise a biguanide and a sodium-dependent glucose transporter (SGLT2) inhibitor; and optionally at least one pharmaceutically acceptable excipient;
wherein at least one active agent is in slow release form.

2. The pharmaceutical composition of claim 1, wherein the said composition comprises a slow release biguanide and an immediate release sodium-dependent glucose transporter (SGLT2) inhibitor.

3. The pharmaceutical composition of claim 1, wherein the said composition comprises a slow release sodium-dependent glucose transporter (SGLT2) inhibitor and a slow release biguanide.

4. The pharmaceutical composition of claim 1, wherein the said composition comprises a slow release sodium-dependent glucose transporter (SGLT2) inhibitor and an immediate release biguanide.

5. The pharmaceutical composition of claim 1, wherein the said composition comprises a slow release biguanide, a slow release sodium-dependent glucose transporter (SGLT2) inhibitor and an immediate release layer comprising a sodium-dependent glucose transporter (SGLT2) inhibitor.

6. The pharmaceutical composition of claim 1, wherein the biguanide is metformin.

7. The pharmaceutical composition of claim 1, comprising a slow release metformin and a slow release sodium-dependent glucose transporter inhibitor and optionally at least one pharmaceutically acceptable excipient;
wherein the said composition exhibits the following dissolution profile: after about 2 hours 0 to about 25% of the metformin is released; after about 4 hours about 20 to about 45% of the metformin is released; after about 8 hours about 45 to about 90% of the metformin is released, after 12 hours not less than 60% of the metformin is released; and after 16 hours not less than 70% of the metformin is released;
when tested in a USP type 2 apparatus, paddle, at 75 rpms in 900 ml of simulated intestinal fluid, pH 7.5 phosphate buffer and at 37° C.

8. The pharmaceutical composition of claim 1, comprising a slow release metformin and a slow release sodium-dependent glucose transporter inhibitor and optionally at least one pharmaceutically acceptable excipient,
wherein the said pharmaceutical composition exhibits the following dissolution profile: after 2 hours 0-25% of the DPP IV inhibitor is released; after about 4 hours about 20 to about 55% of the SGLT2 inhibitor is released; after about 8 hours about 45 to about 90% of the SGLT2 inhibitor is released; and
after about 12 hours at least about 80% of the SGLT2 inhibitor is released;
and after about 16 hours at least about 90% of the SGLT2 inhibitor is released;
when tested in a USP type 2 apparatus, paddle, at 75 rpms in 900 ml of simulated intestinal fluid, pH 7.5 phosphate buffer and at 37° C.

9. The pharmaceutical composition of claim 1, comprising a slow release metformin and an immediate release sodium-dependent glucose transporter inhibitor and optionally at least one pharmaceutically acceptable excipient,
wherein the said composition exhibits the following dissolution profile after about 2 hours, from about 7% to about 60% of the metformin is released; after about 4 hours, from about 15% to about 90% of the metformin is released; after about 8 hours, from about 50% to about 100% of the metformin is released; and
after about 12 hours, at least about 75% of the metformin is released;
when tested in 900 ml of phosphate buffer pH 6.8, at 75 rpm, in USP Apparatus I.

10. The pharmaceutical composition of claim 1, wherein the said composition is suitable for once daily administration.

11. A method for treating diabetes comprising administering, to a patient in need thereof, a pharmaceutical composition of claim 1, comprising a biguanide and a sodium-dependent glucose transporter (SGLT2) inhibitor and optionally at least one pharmaceutically acceptable excipient, wherein at least one active agent is in slow release form.

12. The method of claim 11, wherein the said pharmaceutical composition comprises a slow release sodium-dependent glucose transporter (SGLT2) inhibitor and a slow release biguanide and at least one pharmaceutically acceptable excipient.

13. The method of claim 11, wherein the said pharmaceutical composition comprises a slow release sodium-dependent glucose transporter (SGLT2) inhibitor is in slow release form and an immediate release biguanide.

14. The method of claim 11, wherein the said pharmaceutical composition wherein the said composition comprises a slow release biguanide, a slow release sodium-dependent glucose transporter (SGLT2) inhibitor and an immediate release layer comprising a sodium-dependent glucose transporter (SGLT2) inhibitor, and at least one pharmaceutically acceptable excipient.

15. A pharmaceutical kit comprising pharmaceutical composition of claim 1,
wherein the composition comprises a slow release biguanide and an immediate release sodium-dependent glucose transporter (SGLT2) inhibitor and optionally at least one pharmaceutically acceptable excipient.

16. The pharmaceutical kit of claim 11, comprising a formulation of a slow release sodium-dependent glucose transporter (SGLT2) inhibitor and a slow release biguanide.

17. The pharmaceutical kit of claim 11, comprising a formulation of a slow release biguanide; a slow release sodium-dependent glucose transporter (SGLT2) inhibitor and an immediate release layer comprising biguanide.

18. A pharmaceutical kit of claim 11, comprising a slow release formulation of a slow release sodium-dependent glucose transporter (SGLT2) inhibitor and a slow release biguanide, and an immediate release layer comprising a sodium-dependent glucose transporter (SGLT2) inhibitor.
